SEQUENCE LISTING (29 sequences)

SEQ ID NO: 1 - protein, 9 aa, Artificial Sequence
  Synthetic
GLRILLLKV

SEQ ID NO: 2 - protein, 10 aa, Artificial Sequence
  Synthetic
KLLLLLRLGV

SEQ ID NO: 3 - protein, 10 aa, Artificial Sequence
  Synthetic
KLKLKLKLGV

SEQ ID NO: 4 - protein, 10 aa, Artificial Sequence
  Synthetic
LRLLLLLKVG

SEQ ID NO: 5 - protein, 10 aa, Artificial Sequence
  Synthetic
LRLLLLLKVG

SEQ ID NO: 6 - protein, 10 aa, Artificial Sequence
  Synthetic
LRLLLLLKVG

SEQ ID NO: 7 - protein, 10 aa, Artificial Sequence
  Synthetic
LKLLLLLKVG

SEQ ID NO: 8 - protein, 10 aa, Artificial Sequence
  Synthetic
LRLLLLLKVG

SEQ ID NO: 9 - protein, 10 aa, Artificial Sequence
  Synthetic
LLLRLLLKVG

SEQ ID NO: 10 - protein, 10 aa, Artificial Sequence
  Synthetic
RLLLLLLKVG

SEQ ID NO: 11 - protein, 10 aa, Artificial Sequence
  Synthetic
LLLLLRLKVG

SEQ ID NO: 12 - protein, 10 aa, Artificial Sequence
  Synthetic
LRLKLLVLLG

SEQ ID NO: 13 - protein, 10 aa, Artificial Sequence
  Synthetic
LRLLLLLRVG

SEQ ID NO: 14 - protein, 10 aa, Artificial Sequence
  Synthetic
LKLKLKLKVG

SEQ ID NO: 15 - protein, 10 aa, Artificial Sequence
  Synthetic
LRLLLKLKVG

SEQ ID NO: 16 - protein, 10 aa, Artificial Sequence
  Synthetic
LRLKLLLKVG

SEQ ID NO: 17 - protein, 10 aa, Artificial Sequence
  Synthetic
LRLKLKLKVG

SEQ ID NO: 18 - protein, 10 aa, Artificial Sequence
  Synthetic
LKLKKKLKVG

SEQ ID NO: 19 - protein, 10 aa, Artificial Sequence
  Synthetic
LRLKKKLKVG

SEQ ID NO: 20 - protein, 10 aa, Artificial Sequence
  Synthetic
LKLKKKKKVG

SEQ ID NO: 21 - protein, 10 aa, Artificial Sequence
  Synthetic
LRLKKKKKVG

SEQ ID NO: 22 - protein, 6 aa, Artificial Sequence
  Synthetic
LRLKVG

SEQ ID NO: 23 - protein, 8 aa, Artificial Sequence
  Synthetic
LRLLLKVG

SEQ ID NO: 24 - protein, 12 aa, Artificial Sequence
  Synthetic
LSLRLLLLLKVG

SEQ ID NO: 25 - protein, 12 aa, Artificial Sequence
  Synthetic
LKLRLLLLLKVG

SEQ ID NO: 26 - protein, 10 aa, Artificial Sequence
  Synthetic
KLKLLLRLGV

SEQ ID NO: 27 - protein, 10 aa, Artificial Sequence
  Synthetic
RLLLLLRLGV

SEQ ID NO: 28 - protein, 10 aa, Artificial Sequence
  Synthetic
LLLLLLLLVG

SEQ ID NO: 29 - protein, 5 aa, Artificial Sequence
  Synthetic
ILLLK

(19) United States
(12) Patent Application Publication
(10) Pub. No.: US 2012/0077732 A1
(43) Pub. Date: Mar. 29, 2012

(54) CYCLIC PEPTIDES AND USES THEREOF

(75) Inventors: Nicholas Manolios, Kensington (AU); Veronika Judit Bender, Cremorne (AU); Marina Ali, Parramatta (AU)

(73) Assignee: SYDNEY WEST AREA HEALTH SERVICE, Westmead, New South Wales (AU)

(21) Appl. No.: 13/130,718

(22) PCT Filed: Nov. 24, 2009
(86) PCT No.: PCT/AU09/01530

§ 371 Date: Dec. 13, 2011

(30) Foreign Application Priority Data
Nov. 24, 2008 (AU) 2008906085

(51) Int. Cl. A61K 38/12; C07K 16/44; A61P 37/06; A61P 25/00; A61P 5/00; A61P 9/00; A61P 11/06; A61P 11/00; A61P 17/00; A61P 17/10; A61P 31/04; C07K 9/00; A61P 3/08; C07K 17/02; A61P 3/10; A61P 31/00; A61P 35/00; A61P 9/12; C07K 7/64

(52) U.S. Cl. 514/1.5; 530/321; 514/21.1; 530/389.1; 514/1.7; 514/6.9; 514/6.8; 514/2.3; 514/19.9; 514/17.7; 514/18.6; 514/15.7; 514/4.2

(57) ABSTRACT

The present invention relates to cyclic peptides, comprising alternating D- and L- amino acids and wherein the peptide possesses immunomodulatory activity. The present invention also relates to pharmaceutical compositions comprising the cyclic peptides and to methods for the treatment of disease.

### TECHNICAL FIELD

The present invention relates to synthetic cyclic peptides. The present invention also relates to uses of these peptides, in particular for treating diseases and/or conditions associated with inhibition of T-cell activation or function, allergic airways disease, microbial infections and cancer.

### BACKGROUND

A vast number of diseases and conditions are associated with T-cell function or activation. Accordingly, there is an ever present need for medicinal agents with immunomodulatory activity capable of mediating the activation and/or function of T-cells.

Peptides, either artificial in sequence or representing fragments of larger polypeptides, offer one possibility as therapeutic agents. The present inventors have previously shown that a linear peptide, designated Core Peptide (CP), acts as an immunomodulatory agent and inhibits is IL-2 production in T-cells following antigen recognition (Manolios et. al., 1997). In vivo, CP has been shown to reduce T-cell mediated inflammation in animal models of adjuvant induced arthritis, allergic encephalomyelitis and delayed type contact hypersensitivity. CP is 9 amino acids in length (GLRILLLKV), the sequence of which is derived from the T-cell antigen receptor (TCR-α) transmembrane region.

However a difficulty in employing linear peptides therapeutically is their delivery to the desired site of action in an intact and biologically active form without the peptide being degraded, or integrated into other tissues. In the case of oral peptide delivery, a number of major obstacles in the gastrointestinal tract are encountered and include degradative enzymes and extreme pH, which influence stability and bio-absorption. Most peptides are poorly absorbed, enzyme and pH sensitive, easily biodegradable and have low solubility. Accordingly, there is a need to improve the delivery and stability of peptides to overcome the problems associated with linear molecules.

### SUMMARY

As disclosed herein the present inventors have surprisingly found that various synthetic cyclic peptides with alternating D- and L-amino acids have improved efficacy and specificity compared to CP, whilst lending the same, or greater, biological efficacy as CP. By creating a cyclic peptide, oral delivery and pH stability are improved and enzyme degradation reduced, increasing the utility of the compound.

According to a first aspect of the present invention there is provided a cyclic peptide comprising alternating D- and L-form amino acids, and wherein the peptide possesses immunomodulatory activity.

The immunomodulatory activity may comprise the ability to inhibit T cell activation, proliferation or stimulation.

The cyclic peptide may comprise between 6 and 14 amino acids. The cyclic peptide may comprise between 8 and 12 amino acids. In one embodiment of the first aspect, the cyclic peptide comprises 10 amino acids, in which 5 amino acids are L-form amino acids and 5 amino acids are D-form amino acids.

The cyclic peptide may have a net positive charge. The net positive charge may be at least +2.

The peptide may comprise two positively charged amino acid residues separated by 1 to 5 hydrophobic residues. At least one of the positively charged residues may be a lysine residue.

In one embodiment, the cyclic peptide comprises a lysine residue and a second positively charged residue separated by a spacer of five amino acid residues, wherein the second positively charged residue is an arginine or lysine residue. These amino acids may be D- or L-form amino acids. The five amino acid spacer may comprise no net charge or a net positive charge.

A cyclic peptide of the invention may comprise a structure as depicted in any one FIGS. 1 to 4.

According to a second aspect of the present invention there is provided a cyclic peptide (C1) of formula I:

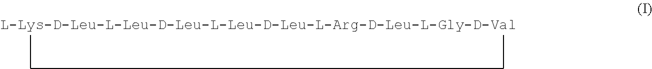

According to a third aspect of the present invention there is provided a cyclic peptide of formula II:

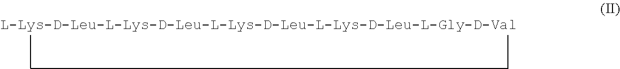

According to a fourth aspect of the present invention there is provided a cyclic peptide selected from the group consisting of, L_{D}RL_{D}LL_{D}LL_{D}KV_{D}G, L_{D}R_{D}L_{D}L_{D}L_{D}L_{D}L_{D}K_{D}V_{D}G, LRLLLLLKVG, L_{D}KL_{D}LL_{D}LL_{D}KV_{D}G, LR_{D}LL_{D}LL_{D}LK_{D}VG_{D}, L_{D}LL_{D}RL_{D}LL_{D}KV_{D}G, R_{D}LL_{D}LL_{D}LL_{D}KV_{D}G, L_{D}LL_{D}LL_{D}RL_{D}KV_{D}G, L_{D}RL_{D}KL_{D}LV_{D}LL_{D}G, L_{D}RL_{D}LL_{D}LL_{D}RV_{D}G, L_{D}KL_{D}KL_{D}KL_{D}KV_{D}G, L_{D}RL_{D}LL_{D}KL_{D}KV_{D}G, L_{D}RL_{D}KL_{D}LL_{D}KV_{D}G, L_{D}RL_{D}KL_{D}KL_{D}KV_{D}G, L_{D}KL_{D}KK_{D}KL_{D}KV_{D}G, L_{D}RL_{D}KK_{D}KL_{D}KV_{D}G, L_{D}KL_{D}KK_{D}KK_{D}KV_{D}G, L_{D}RL_{D}KK_{D}KK_{D}KV_{D}G, L_{D}RL_{D}KV_{D}G, L_{D}RL_{D}LL_{D}KV_{D}G, GLRILLLKV, L_{D}SL_{D}RL_{D}LL_{D}LL_{D}KV_{D}G, L_{D}KL_{D}RL_{D}LL_{D}LL_{D}KV_{D}G

A cyclic peptide of the first, second, third or fourth aspect may be conjugated to one or more chemical moieties. Conjugation may be via linkage at one or more carboxyl, hydroxyl and/or amide groups. The chemical moiety may be a polypeptide, peptide, lipid, sugar or other chemical compound. The chemical moiety may be a carrier or a therapeutic agent.

According to a fifth aspect of the present invention there is provided a pharmaceutical composition comprising a peptide of any one of the first, second, third or fourth aspects. The pharmaceutical composition may include one or more pharmaceutically acceptable carriers, adjuvants or diluents.

According to a sixth aspect of the present invention there is provided an antibody that selectively binds to a cyclic peptide of any one of the first, second, third or fourth aspects.

According to a seventh aspect the present invention provides a method for treating or preventing a disease state in a subject, said method comprising administering to the subject an effective amount of a peptide of any one of the first, second, third or fourth aspects or a pharmaceutical composition of the fourth aspect. Typically the disease is an auto-immune disease. The disease may be neural, endocrinal, skeletal, dermal, gastrointestinal, cardiac, respiratory, vascular, of the immune system or a transplantation disease. The disease may be a cancer. The disease may be an airway disease such as asthma, chronic eosinophilic pneumonia, COPD, COPD associated with chronic bronchitis, pulmonary emphysema, dyspnea associated or not associated with COPD, COPD characterized by irreversible, progressive airway obstruction, adult respiratory distress syndrome (ARDS), exacerbation of airway hyper-reactivity consequent to other drug therapy, airway disease associated with pulmonary hypertension, rhinitis, bronchial oedema, bronchial asthma, pulmonary oedema, anaphylaxis or angioedema.

According to a eighth aspect the present invention provides a method for treating or preventing a disease state in a subject in which the inhibition of T-cell activation, proliferation or function is desirable, said method comprising administering to the subject an effective amount of a peptide of any one of the first, second, third or fourth aspects or a pharmaceutical composition of the fourth aspect. The inhibition of T-cell activation, proliferation or function may involve the inhibition of IL-2 production in T-cells.

According to a ninth aspect the present invention provides a method for treating or preventing a microbial infection in a subject, said method comprising administering to the subject a therapeutically effective amount of a peptide of any one of the first, second, third or fourth aspects or a pharmaceutical composition of the fifth aspect.

In one embodiment the infection may be a Staphylococcus infection. In a preferred embodiment the Staphylococcus infection may be a Staphylococcus aureus infection. In another embodiment the microbial infection is associated with increased IL-13. The microbial infection may be a parasitic infection. In some embodiments the parasitic infection may be an intracellular parasite. In a preferred embodiment the intracellular parasite may be bacterium or a protozoan. The bacterium may be selected from the group comprising Chlamydia, Rickettsia, Coxiella or Mycobacterium. The Mycobacterium may be Mycobacterium leprae or Mycobacterium tuberculosis. The Protozoan may be selected from the group comprising Plasmodium sp, Leishmania sp, Toxoplasma or Trypanosoma.

According to a tenth aspect the present invention provides the use of a peptide of any one of the first, second, third or fourth aspects for the manufacture of a medicament for the treatment or prevention of a disease state in a subject.

According to an eleventh aspect the present invention provides the use of a peptide of any one of the first, second, third or fourth aspects for the manufacture of a medicament for the treatment or prevention of a microbial infection in a subject.

According to an twelfth aspect the present invention provides a method for treating or preventing a disease state in a subject in which the control of blood glucose levels is desirable, said method comprising administering to the subject an effective amount of a peptide of any one of claims first, second, third or fourth aspects or a pharmaceutical composition of the fifth aspect. The disease state may be diabetes.

### BRIEF DESCRIPTION OF THE DRAWINGS

Embodiments of the present invention will now be described, by way of example only, with reference to the accompanying drawings.

FIG. 1. Cyclized structure of peptide C1. Amino acids are numbered from 1 to 10, according to the order of residues provided in the linear single letter amino acid code. D subscript represents a D-amino acid.

FIG. 2. Cyclized structure of peptide C2. Amino acids are numbered from 1 to 10, according to the order of residues provided in the linear single letter amino acid code. D subscript represents a D-amino acid.

FIG. 3. Cyclized structure of peptide C3. Amino acids are numbered from 1 to 10, according to the order of residues provided in the linear single letter amino acid code. D subscript represents a D-amino acid.

FIG. 4. Cyclized structure of peptide C4. Amino acids are numbered from 1 to 10, according to the order of residues provided in the linear single letter amino acid code. D subscript represents a D-amino acid.

FIG. 5. Antigen presentation assay. % IL-2 produced in T-cells at varying concentrations (μM) of peptides C1-C4 and the linear form of C1 (C1-L).

FIG. 6. Viability and proliferation assay, % viability and proliferation of 2B4 cells in the presence of C1 at 50 μM, relative to a control of 0.5% DMSO.

FIG. 7. Antigen presentation assay. % IL-2 produced, relative to a DMSO control (0.5%) under various T-cell stimulants in the presence of C1 at 50 μM.

FIG. 8. The effect of C1, CP and cyclosporin on total foot swelling (%). Filled circles, placebo treated rats (n=6); crosses, CP treated rats (n=5); open circles, cyclosporine treated rats (n=5); filled triangles, C1 treated rats (n=4).

FIG. 9. ³¹P NMR spectrum of C1 in DMPC/DMPG lipid bilayers (upper).

FIG. 10. ²H NMR spectrum of C1 in DMPC-d54/DMPG bilayers (upper).

FIG. 11. Comparative binding of peptide analogues (50 μM) to DMPC (A) and DMPG (B) liposomes immobilised onto L1 sensor chip.

FIG. 12. Transmission Electron micrograph of C1 (100 μM in Water/HCl) showing rectangular structures.

FIG. 13. Mass spectrum of crude C1 after cyclization with PyPOB indicating the presence of Cl (peak at 1119).

FIG. 14. Mass spectrum of crude C1 after cyclization with PyPQB. Protected C1 was purified by extraction before removing the protecting groups.

FIG. 15. Mass spectrum of crude C1 after cyclization with FDPP indicating the presence of C1 (peak at 1119).

FIG. 16. Schematic diagram of a conventional HPLC system

FIG. 17. Schematic diagram of an at-column dilution system

FIG. 18. Mass spectrum of C1

FIG. 19. Effect of cyclic C1 peptide administration on bronchoalveolar lavage fluid (BALF) inflammatory cell infiltrates in allergic mice: total white blood cells. Data represents the mean+SEM for a minimum of 4 mice per group compared to historical naive, PBS/OVA and OVA/OVA mice. Student's unpaired t-test ***P<0.001, **p<0.005,* p<0.05 compared to OVA/OVA.

FIG. 20: Effect of peptide administration on bronchoalveolar lavage fluid (BALF) inflammatory cell infiltrates in allergic mice: differential white blood cell counts. Data represents the mean+SEM for a minimum of 4 mice per group compared to historical naïve, PBS/OVA and OVA/OVA mice. Student's unpaired t-test ***P<0.001, **p<0.005,* p<0.05 compared to OVA/OVA.

FIG. 21: The effect of peptide administration on bronchoalveolar lavage fluid (BALF) inflammatory cell infiltrates in allergic mice: total white blood cells. Data represents the mean+SEM for a minimum of 7 mice per group. Student's unpaired t-test ***P<0.001, **p<0.005,* p<0.05 compared to OVA/OVA.

FIG. 22: The effect of peptide administration on bronchoalveolar lavage fluid (BALF)inflammatory cell infiltrates in allergic mice: differential white blood cell counts. Data represents the mean+SEM for a minimum of 6 mice per group. Student's unpaired t-test ***P<0.001, **p<0.005,* p<0.05 compared to OVA/OVA.

FIG. 23: The effect of peptide administration on spleen and peribronchial lymph node OVA-specific Th2 cytokine production. Data represents the mean+SEM for a minimum of 6 mice per group. Student's unpaired t-test ***P<0.001, **p<0.005,* p<0.05 compared to OVA/OVA.

FIG. 24: The effect of peptide administration on airways hyperreactivity. Airway resistance (RL) is presented as a percentage of the baseline reactivity to saline in the absence of cholinergic stimuli and represent the mean+SEM for a minimum of 5 mice per group. 2Way ANOVA ***P<0.001, **p<0.005,* p<0.05 compared to OVA/OVA.

FIG. 25: The effect of peptide administration on airways hyperreactivity. Dynamic compliance (CDyn) is presented as a percentage of the baseline reactivity to saline in the absence of cholinergic stimuli and represent the mean+SEM for a minimum of 5 mice per group. 2Way ANOVA ***P<0.001, **p<0.005,* p<0.05 compared to OVA/OVA.

FIG. 26: Blood glucose levels of NOD mice over time after the administration of C1

FIG. 27: Blood glucose levels of NOD mice over time after the administration of water.

### DEFINITIONS

As used herein the term “peptide” means a polymer made up of amino acids linked together by peptide bonds.

The term “analogue” as used herein with reference to a peptide means a peptide which is a derivative of a cyclic peptide of the invention which derivative comprises addition, deletion, substitution of one or more amino acids, such that the peptide retains substantially the same function as the cyclic peptide of the invention.

As used herein the term “treatment”, refers to any and all uses which remedy a disease state or symptoms, prevent the establishment of disease, or otherwise prevent, hinder, retard, or reverse the progression of disease or other undesirable symptoms in any way whatsoever.

As used herein the term “effective amount” includes within its meaning a non-toxic but sufficient amount of an agent or compound to provide the desired therapeutic effect. The exact amount required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

In the context of this specification, the terms “subject” and “patient” are used interchangeably and include humans and individuals of any species of social, economic or research importance including but not limited to members of the genus ovine, bovine, equine, porcine, feline, canine, primates, rodents. In preferred embodiments the patient will be a mammal and more preferably the patient will be a human.

In the context of this specification, the term “comprising” means “including principally, but not necessarily solely”. Furthermore, variations of the word “comprising”, such as “comprise” and “comprises”, have correspondingly varied meanings.

### DETAILED DESCRIPTION

Amino acids can take two mirror forms, the L-isomer or the D-isomer. Naturally occurring amino acids in mammalian polypeptides and proteins contain L-isomer amino acids only. Core Peptide (CP), derived from the transmembrane region of the T-cell antigen receptor (TCR-α) consists of 9 L-isomer amino acids and has the ability to inhibit T-cell antigen specific activation in vitro and in vivo.

Gerber et. al. (2005) compared the activity of the two stereoisomers of CP, that is, CP consisting of L-amino acids only and CP consisting of D-amino acids only. Gerber et al. showed that both the L- and D- forms of CP possessed similar inhibitory activity of T-cell proliferation in vitro. Furthermore both the L- and D-form of CP were equally active in alleviating adjuvant arthritis in vivo.

The present inventors have now found that that the unique cyclic peptide of formula I,

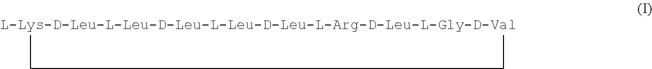

consisting of alternating D- and L-amino acids displays the same biological function as CP. As disclosed herein, in an in vitro antigen presentation assay, cyclic peptides of formula I (above) and formula II,

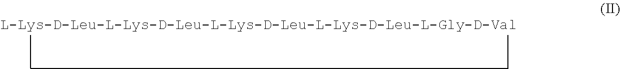

each significantly inhibited T-cell activation as measured by IL-2 production.

The cyclic peptide of formula I possesses not only desirable biological activity, including the inhibition of T-cell activation and antimicrobial activity, but also possesses desirable stability characteristics, increasing the utility of the compound.

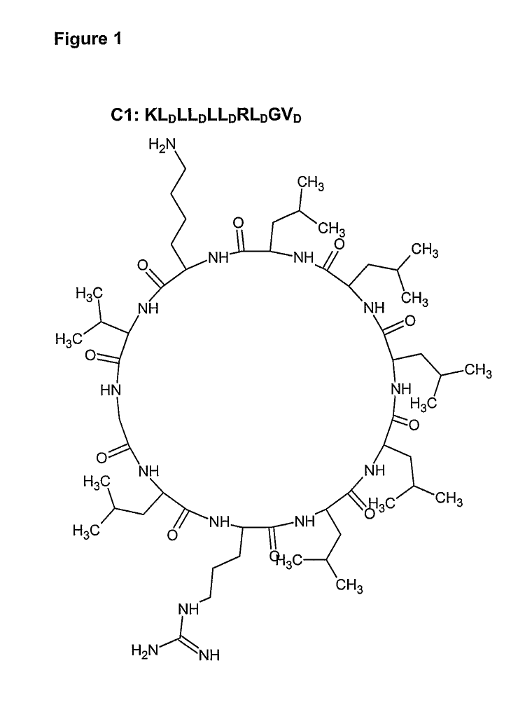

The present invention provides isolated cyclic peptides (C1-C4) having the cyclized structures as depicted in FIGS. 1 to 4 respectively, represented below by the formulas I to II respectively.

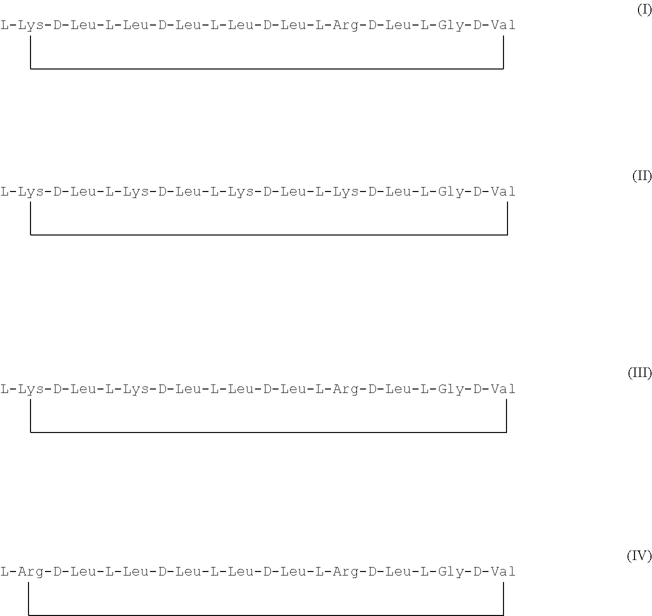

The present invention also provides cyclized structures represented below by the formulas v to xxvii respectively:

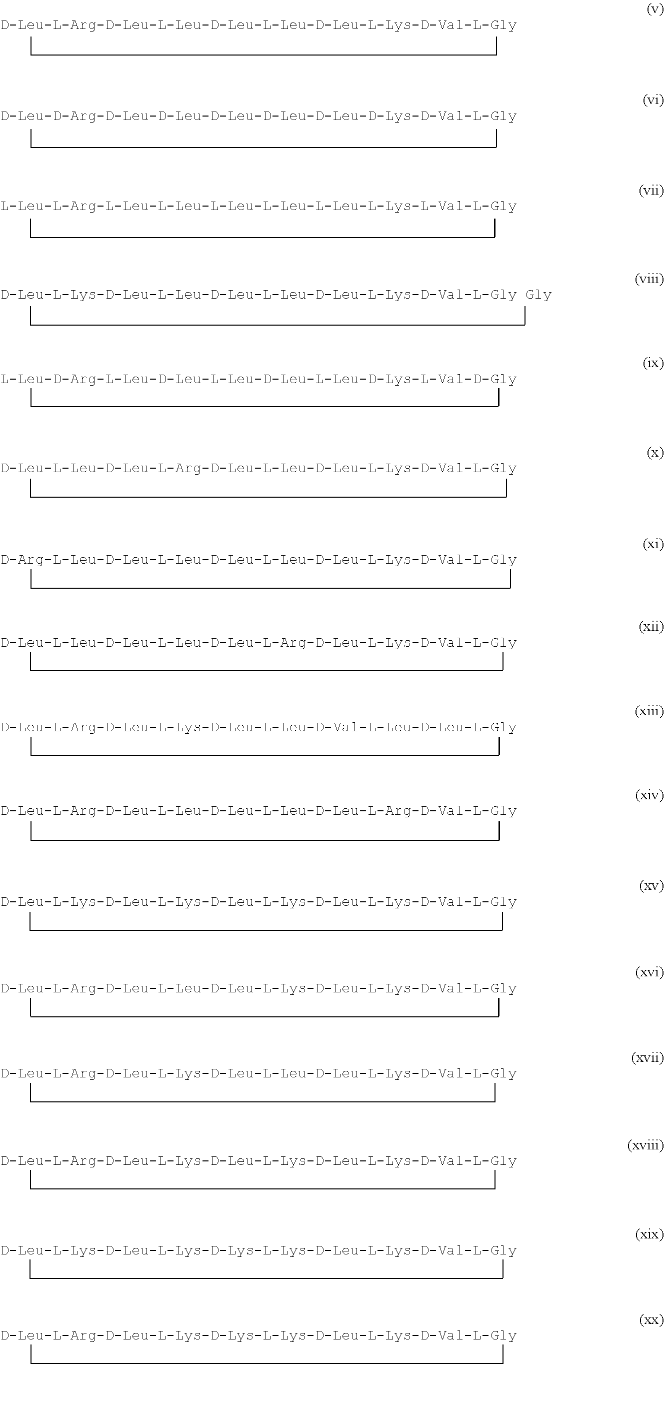

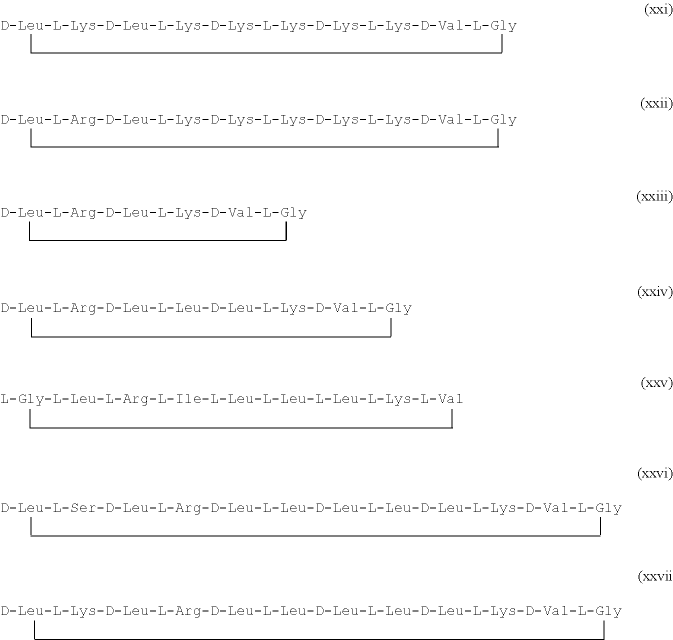

Those skilled in the art will readily appreciate that modifications may be made to the peptides of the invention without departing from the scope of the invention. For example, the particular residues which are in D-form and those in L-form may be altered so long as the D/L chirality is maintained. Additionally, the hydrophobic residues may be replaced by other hydrophobic residues. Further, isosteric replacements may be made to one or more amide bonds between adjacent amino acids in the peptide.

The length of the peptide may also be altered. Typically, peptides of the invention comprise between 6 and 14 amino acids, between 7 and 13 amino acids, between 8 and 12 amino acids, between 9 and 11 amino acids, or more typically 10 amino acids.

The present invention also contemplates peptide conjugates wherein peptides of the invention are linked to one or more additional chemical moieties. Conjugation may be via linkage at one or more carboxyl, hydroxyl and/or amide groups. By way of example, the chemical moiety may be a polypeptide, peptide, lipid, sugar or other chemical compound. The chemical moiety may be a carrier agent, facilitating or enhancing the in vivo delivery of the peptide and/or targeting and delivering the peptide to the region, tissue or cell in which activity of the peptide is desired. Suitable carrier molecules include lipids, such as short or long chain fatty acids. Alternatively, the chemical moiety may be a therapeutic agent such as a pharmaceutical compound.

Cyclic D-, L-α-peptides are proteolytically stable, easy to synthesise, and can be derived from a vast potentially membrane-active sequence space. The unique abiotic structure of the cyclic peptides and their rapid bactericidal action may also limit temporal acquirement by drug resistant bacteria. The low molecular weight D, L-α-peptides disclosed herein therefore offer an attractive complement to the current arsenal of naturally derived antibiotics, and hold considerable potential in combating a variety of existing and emerging infectious diseases.

For example, as disclosed herein the cyclic peptide of formula I (C1) is an active biological compound with potent immunosuppression. Biophysical studies indicate that C1 does not damage the bilayer of model membranes like other cyclic and antimicrobial peptides. The cyclic peptide C1 is stable over a large pH range (1-11) and when incubated with trypsin. The ability of C1 to inhibit immune reactions provides a new and exciting alternative to existing therapies.

Peptides of the present invention may be synthesised first as linear molecules by standard methods of liquid or solid phase chemistry well known to those of ordinary skill in the art. For example such molecules may be synthesised following the solid phase chemistry procedures of Steward and Young (Steward, J. M. & Young, J. D., Solid Phase Peptide Synthesis. (2nd Edn.) Pierce Chemical Co., Illinois, USA (1984). Linear peptides can then be cyclized using techniques and procedures also well known to those of skill in the art.

In particular embodiments a linear peptide of the invention may be cyclized in accordance with the following method. A solution of a peptide of the invention in dichloromethane (DCM; ˜1 mM) may be stirred with (benzotriazol-1-yloxy)tripyrrolidinophosphonium hexafluorophosphate (PyBOP; 4 eq.) and N,N-diisopropylethylamine (DIEA; 10 eq.) overnight. Cyclized peptide may then be dried on a rotary evaporator prior to deprotection with a mixture of 95% trifluoroacetic acid (TFA):2.5% water:and 2.5% triisopropylsilane (TIPSI). Crude peptide may be characterized by mass spectrometry.

In another embodiment cyclization may be performed as described above but the protected cyclized peptide may be first precipitated in water and the precipitate washed with 0.5M sodium hydrogen carbonate (NaHCO₃), ether and acetonitrile respectively. Protecting groups may then be removed in TFA/scavenger mixture. The peptide may be characterized by mass spectrometry.

In another embodiment cyclization may be performed under anhydrous conditions, 1.5 equiv of pentafluorophenyl diphenylphosphinate (FDPP; 39 mg, 0.1 mmol) may be added to a solution of linear protected C1 (100 mg, 0.007 mmol) in acetonitrile (14 ml), followed by addition of 3 equiv of DIEA (0.2 mmol). The reaction mixture may then be stirred at room temperature overnight, after which the solvent may be removed by rotary evaporation prior to characterization by mass spectrometry.

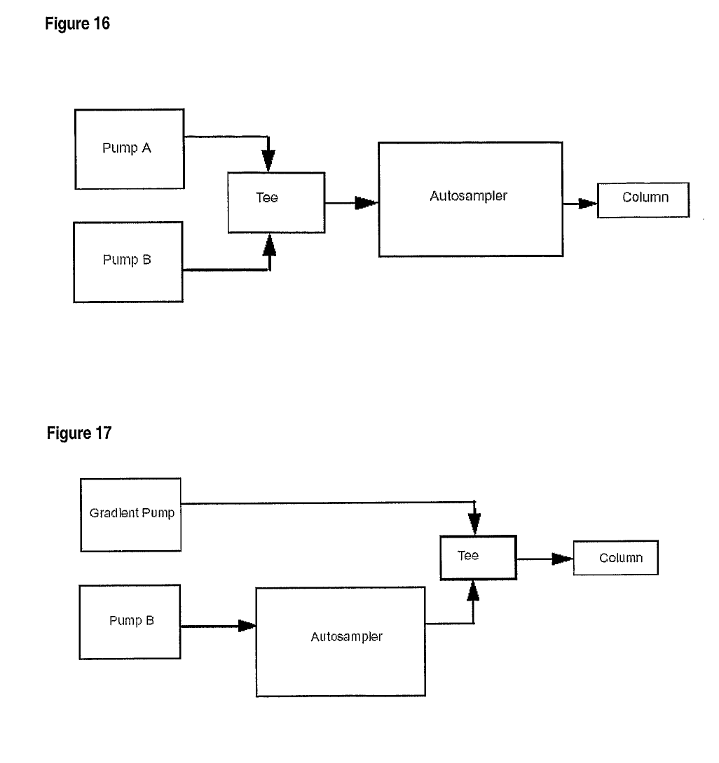

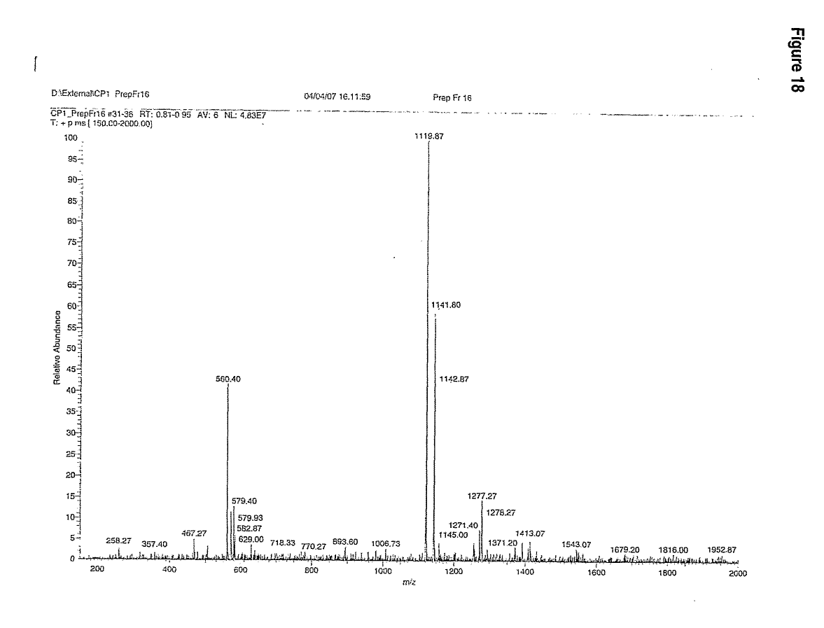

Purification of a cyclic peptide of the invention may be performed by HPLC. Typically the hydrophobic nature of the cyclised peptides of the invention have made purification of the peptides difficult using conventional HPLC techniques. The inventors have used an At-Column Dilution (ACD) technique to purify the peptides using HPLC. This technique was developed for injecting relatively large volumes of strong sample diluents. ACD technique also prevents bulk precipitation in the sample loop or in the column itself. FIGS. 16 and 17 are schematic diagrams of conventional and ACD HPLC techniques.

For ACD purification a peptide of the invention may be dissolved in approximately 0.5 ml of acetonitrile, 200 μl of 10% TFA. DMSO may then be added until a clear solution is obtained. The sample may be centrifuged prior to collecting the supernatant for loading on to a reverse phase column, such as a c18 column. The sample may be loaded using standard ACD techniques. A peptide of the invention may be loaded on to the column at a flow rate of, for example, 0.1 ml/min. Simultaneously the mobile phase may be pumped through the column using another pump at a flow rate of, for example, 5 ml/min. The presence of a peptide of the invention in fraction collected from the column may be confirmed by mass spectrometry.

Therapeutic and Prophylactic Applications

Peptides of the present invention find application in the treatment or prevention of a variety of diseases and conditions such as diseases in which inhibition of T-cell function is desirable, particularly auto-immune diseases and cancers. By way of example, diseases and disorders to which methods and compositions of the present invention are applicable include, but are not limited to, neural diseases such as multiple sclerosis and Guillain Barre Syndrome, endocrine diseases such as diabetes, Hashimotos disease and pernicious anaemia, skeletal diseases such as rheumatoid arthritis, ankylosing spondylitis, reactive arthritis and systemic lupus erythematosus, immune diseases such as transplant rejection syndrome, urticaria and drug allergy, dermal diseases such as pemphigus, eczema, contact dermatitis and psoriasis, gastrointestinal tract diseases such as ulcerative colitis and Crohn's disease, respiratory diseases such as asthma and pneumonitis, transplantation disease, cardiac diseases, vascular diseases and cancer.

Interleukin-5 (IL-5) is associated with allergic diseases including allergic rhinitis and asthma which are characterised by increased eosinophils in the circulation, in airway tissue and in sputum. Interleukin 13 (IL-13) is associated with the induction of airway disease, however it also induces physiological changes in parasitised organs for example in the gut it induces contractions and hyper-secretion of glycoprotein from epithelial cells. Parasitic infection often leads to granulomas the formation of which is at least partly controlled by IL-13. Granulomas result in organ damage and thus profound or sometimes fatal disease rather than resolution of the infection. IL-13 is thought to antagonize Th1 responses required to resolve intracellular infections. In this context of immune dysregulation which is characterised by recruitment of large numbers of Th2 cells IL-13 inhibits the destruction of intracellular pathogens by the hosts immune system.

IL-13 also induces features of allergic lung disease, including airway hyperresponsiveness, goblet cell metaplasia and mucus hypersecretion which contribute to airway obstruction.

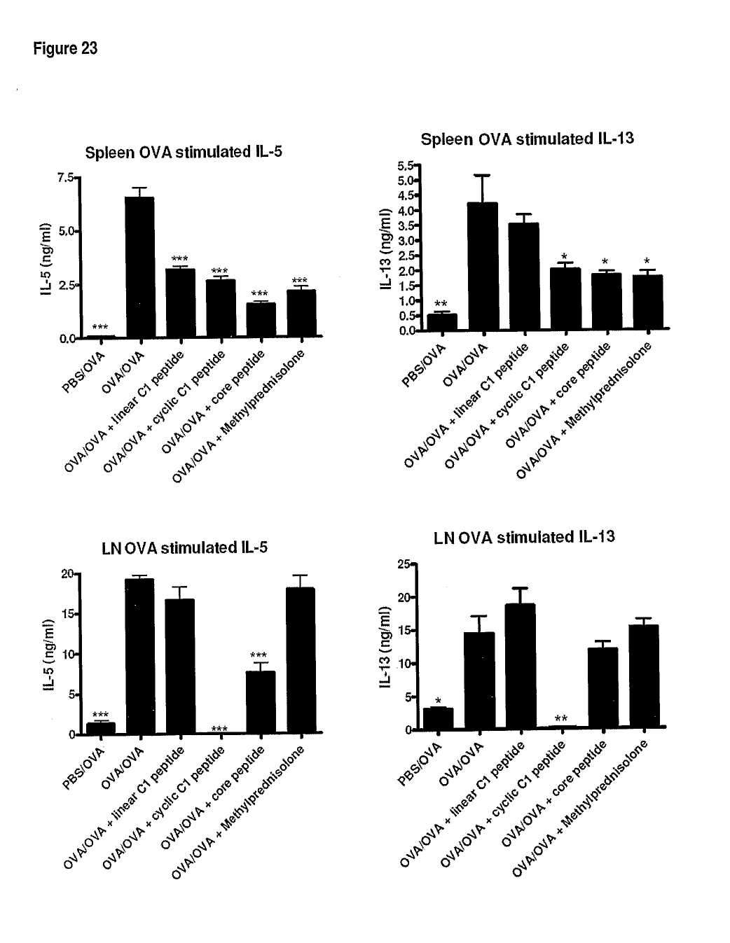

As peptides of the present invention reduce IL-5 and IL-13 levels (See for example FIG. 23) the peptides of the present invention find application in the treatment of diseases characterised by increased IL-5 or IL-13 or both and where the increase in IL-5 and/or IL-13 contributes to the pathology of the disease. Peptides of the present invention also find application in the inhibition of T-cell function for the treatment or prevention of a variety of diseases and conditions such as non-autoimmune diseases in which the T-cell may be involved by virtue of providing CD4 help. By way of example diseases and disorders to which methods and compositions of the present invention are applicable include, but are not limited to airway diseases, dermal diseases, osteoporosis, atherosclerosis, multiple sclerosis, and ischaemic heart disease.

In particular the airway disease may be an airway disease such as asthma, chronic eosinophilic pneumonia, COPD (chronic obstructive pulmonary disease), COPD associated with bronchitis, pulmonary emphysema, dyspnea associated or not associated with COPD, COPD characterized by irreversible, progressive airway obstruction, adult respiratory distress syndrome (ARDS), exacerbation of airway hyper-reactivity consequent to other drug therapy, airway disease associated with pulmonary hypertension, rhinitis, bronchial oedema, bronchial asthma, pulmonary oedema, anaphylaxis or angioedema.

Examples of dermal diseases include vitiligo, psoriasis, eczema, Herpes simplex, erythema nodosum, acne vulgaris, erythema, erythema multiforme, neurodermatitis, drug rash, urticaria, contact dermatitis, dermatomycosis and herpes zoster.

Methods and compositions of the present invention also find application in the treatment of a variety of infectious diseases and microbial infections. Such infections may result from, or be associated with, either Gram-negative or Gram-positive bacteria.

Those skilled in the art will appreciate that peptides of the invention may be administered alone or in conjunction with one or more additional agents. For example, a peptide of the invention may be administered together with one or more agents for inhibiting T-cell activation and/or function, such as cyclosporin. In the treatment of microbial infections, a peptide of the invention may be administered in conjunction with one or more antibiotics or antimicrobial agents. Suitable agents which may be used in combination with the peptides of the present invention will be known to those of ordinary skill in the art.

For such combination therapies, each component of the combination may be administered at the same time, or sequentially in any order, or at different times, so as to provide the desired effect. When administered separately, it may be preferred for the components to be administered by the same route of administration, although it is not necessary for this to be so. Alternatively, the components may be formulated together in a single dosage unit as a combination product. Suitable agents which may be used in combination with the compositions of the present invention will be known to those of ordinary skill in the art.

Cyclic peptides of the invention, or antibodies thereof, may be administered in the form of pharmaceutical compositions either therapeutically or preventively. In a therapeutic application, compositions are administered to a patient already suffering from a disease, in an amount sufficient to cure or at least partially arrest the disease and its complications. The composition should provide a quantity of the active agent sufficient to effectively treat the patient.

The effective dose level for any particular patient will depend upon a variety of factors including: the disorder being treated and the severity of the disorder; activity of the agent employed; the composition employed; the age, body weight, general health, sex and diet of the patient; the time of administration; the route of administration; the rate of sequestration of the agent; the duration of the treatment; drugs used in combination or coincidental with the treatment, together with other related factors well known in medicine.

One skilled in the art would be able, by routine experimentation, to determine an effective, non-toxic amount of agent which would be required to treat applicable diseases.

Generally, an effective dosage is expected to be in the range of about 0.0001 mg to about 1000 mg per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; about 1.0 mg to about 250 mg per kg body weight per 24 hours. More typically, an effective dose range is expected to be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours, about 6.0 mg to about 10 mg per kg body weight per 24 hours or about 1.0 mg to about 6 mg per kg body weight per 24 hours

Alternatively, an effective dosage may be up to about 500 mg/m². Generally, an effective dosage is expected to be in the range of about 25 to about 500 mg/m², preferably about 25 to about 350 mg/m², more preferably about 25 to about 300 mg/m², still more preferably about 25 to about 250 mg/m², even more preferably about 50 to about 250 mg/m², and still even more preferably about 75 to about 150 mg/m².

Typically, in therapeutic applications, the treatment would be for the duration of the disease state.

Further, it will be apparent to one of ordinary skill in the art that the optimal quantity and spacing of individual dosages will be determined by the nature and extent of the disease state being treated, the form, route and site of administration, and the nature of the particular individual being treated. Also, such optimum conditions can be determined by conventional techniques.

It will also be apparent to one of ordinary skill in the art that the optimal course of treatment, such as, the number of doses of the composition given per day for a defined number of days, can be ascertained by those skilled in the art using conventional course of treatment determination tests.

In general, suitable compositions may be prepared according to methods which are known to those of ordinary skill in the art and accordingly may include a pharmaceutically acceptable carrier, diluent and/or adjuvant.

These compositions can be administered by standard routes. In general, the compositions may be administered by the parenteral (e.g., intravenous, intraarticular, intraspinal, subcutaneous or intramuscular), oral or topical route.

The carriers, diluents and adjuvants must be “acceptable” in terms of being compatible with the other ingredients of the composition, and not deleterious to the recipient thereof.

Examples of pharmaceutically acceptable carriers or diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

The compositions of the invention may be in a form suitable for administration by injection, in the form of a formulation suitable for oral ingestion (such as capsules, tablets, caplets, elixirs, for example), in the form of an ointment, cream or lotion suitable for topical administration, in a form suitable for delivery as an eye drop, in an aerosol form suitable for administration by inhalation, such as by intranasal inhalation or oral inhalation, in a form suitable for parenteral administration, that is, subcutaneous, intramuscular or intravenous injection.

For administration as an injectable solution or suspension, non-toxic parenterally acceptable diluents or carriers can include, Ringer's solution, isotonic saline, phosphate buffered saline, ethanol and 1,2 propylene glycol.

Suitable forms for oral administration include tablets, lozenges, pills, capsules, aerosols, elixirs, powders, granules, solutions, suspensions and emulsions. Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration.

Adjuvants typically include emollients, emulsifiers, thickening agents, preservatives, bactericides and buffering agents.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay is agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

The emulsions for oral administration may further comprise one or more emulsifying agents. Suitable emulsifying agents include dispersing agents as exemplified above or natural gums such as guar gum, gum acacia or gum tragacanth.

Methods for preparing parenterally administrable compositions are apparent to those skilled in the art, and are described in more detail in, for example, Remington's Pharmaceutical Science, 15th ed., Mack Publishing Company, Easton, Pa., hereby incorporated by reference herein.

The topical formulations according to the present invention, comprise an active ingredient together with one or more acceptable carriers, and optionally any other therapeutic ingredients. Formulations suitable for topical administration include liquid or semi-liquid preparations suitable for penetration through the skin to the site of where treatment is required, such as liniments, lotions, creams, ointments or pastes, and drops suitable for administration to the eye, ear or nose.

Drops according to the present invention may comprise sterile aqueous or oily solutions or suspensions. These may be prepared by dissolving the active ingredient in an aqueous solution of a bactericidal and/or fungicidal agent and/or any other suitable preservative, and optionally including a surface active agent. The resulting solution may then be clarified by filtration, transferred to a suitable container and sterilised. Sterilisation may be achieved by: autoclaving or maintaining at 90° C.-100° C. for half an hour, or by filtration, followed by transfer to a container by an aseptic technique. Examples of bactericidal and fungicidal agents suitable for inclusion in the drops are phenylmercuric nitrate or acetate (0.002%), benzalkonium chloride (0.01%) and chiorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those described above in relation to the preparation of drops. Lotions or liniments for application to the skin may also include an agent to hasten drying and to cool the skin, such as an alcohol or acetone, and/or a moisturiser such as glycerol, or oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogols.

The composition may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

The compositions may also be administered in the form of liposomes. Liposomes are generally derived from phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq., the contents of which is incorporated herein by reference.

Antibodies

The present invention also provides antibodies that selectively bind to the cyclic peptides of the present invention. Suitable antibodies include, but are not limited to polyclonal, monoclonal, chimeric, humanised, single chain, Fab fragments, and an Fab expression library. Antibodies of the present invention may act as agonists or antagonists of the cyclic peptides of the present invention.

Methods for the generation of suitable antibodies will be readily appreciated by those skilled in the art. For example, a monoclonal antibody, typically containing Fab portions, may be prepared using the hybridoma technology described in Antibodies—A Laboratory Manual, Harlow and Lane, eds., Cold Spring Harbor Laboratory, N.Y. (1988).

In essence, in the preparation of monoclonal antibodies directed toward peptides of the invention, any technique that provides for the production of antibody molecules by continuous cell lines in culture may be used. These include the hybridoma technique originally developed by Kohler et al., Nature, 256:495-497 (1975), as well as the trioma technique, the human B-cell hybridoma technique (Kozbor et al., Immunology Today, 4:72 (1983)), and the EBV-hybridoma technique to produce human monoclonal antibodies (Cole et al., in Monoclonal Antibodies and Cancer Therapy, pp. 77-96, Alan R. Liss, Inc., (1985)). Immortal, antibody-producing cell lines can be created by techniques other than fusion, such as direct transformation of B lymphocytes with oncogenic DNA, or transfection with Epstein-Barr virus. See, e.g., M. Schreier et al., “Hybridoma Techniques” (1980); Hammerling et al., “Monoclonal Antibodies and T-cell Hybridomas” (1981); Kennett et al., “Monoclonal Antibodies” (1980).

In summary, a means of producing a hybridoma from which the monoclonal antibody is produced, a myeloma or other self-perpetuating cell line is fused with lymphocytes obtained from the spleen of a mammal hyperimmunised with a recognition factor-binding portion thereof, or recognition factor, or an origin-specific DNA-binding portion thereof. Hybridomas producing a monoclonal antibody useful in practicing this invention are identified by their ability to immunoreact with the present recognition factor and their ability to inhibit specified transcriptional activity in target cells.

A monoclonal antibody useful in practicing the present invention can be produced by initiating a monoclonal hybridoma culture comprising a nutrient medium containing a hybridoma that secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques.

Similarly, there are various procedures known in the art which may be used for the production of polyclonal antibodies to cyclic peptides of the invention. For the production of polyclonal antibody, various host animals can be immunized by injection with the cyclic peptide, including but not limited to rabbits, mice, rats, sheep, goats, etc. Further, the cyclic peptides can is be conjugated to an immunogenic carrier, e.g., bovine serum albumin (BSA) or keyhole limpet hemocyanin (KLH). Also, various adjuvants may be used to increase the immunological response, including but not limited to Freund's (complete and incomplete), mineral gels such as aluminium hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

Screening for the antibody can also be accomplished by a variety of techniques known in the art. Assays for immunospecific binding of antibodies may include, but are not limited to, radioimmunoassays, ELISAs (enzyme-linked immunosorbent assay), sandwich immunoassays, immunoradiometric assays, gel diffusion precipitation reactions, immunodiffusion assays, in situ immunoassays, Western blots, precipitation reactions, agglutination assays, complement fixation assays, immunofluorescence assays, protein A assays, and immunoelectrophoresis assays, and the like (see, for example, Ausubel et al., eds, 1994, Current Protocols in Molecular Biology, Vol. 1, John Wiley & Sons, Inc., New York). Antibody binding may be detected by virtue of a detectable label on the primary antibody. Alternatively, the antibody may be detected by virtue of its binding with a secondary antibody or reagent which is appropriately labelled. A variety of methods are known in the art for detecting binding in an immunoassay and are within the scope of the present invention.

Antibodies of the present invention can be used in diagnostic methods and kits that are well known to those of ordinary skill in the art.

An antibody raised against a cyclic peptide of the invention has binding affinity for the cyclic peptide. Preferably, the antibody has binding affinity or avidity greater than about 10⁵ M⁻¹, more preferably greater than about 10⁶ M⁻¹, more preferably still greater than about 10⁷ M⁻¹ and most preferably greater than about 10⁸ M⁻¹.

In terms of obtaining a suitable amount of an antibody according to the present invention, one may manufacture the antibody(s) using batch fermentation with serum free medium. After fermentation the antibody may be purified via a multistep procedure incorporating chromatography and viral inactivation/removal steps. For instance, the antibody may be first separated by Protein A affinity chromatography and then treated with solvent/detergent to inactivate any lipid enveloped viruses. Further purification, typically by anion and cation exchange chromatography may be used to remove residual proteins, solvents/detergents and nucleic acids. The purified antibody may be further purified and formulated into 0.9% saline using gel filtration columns. The formulated bulk preparation may then be sterilised and viral filtered and dispensed.

The present invention will now be described with reference to specific examples, which should not be construed as in any way limiting the scope of the invention.

### EXAMPLES

### General Materials and Peptide Synthesis.

Dimyristoyl-L-α-phoshatidyl choline (DMPC), dimyristoyl-L-α-phosphatidyl-DL-glycerol (DMPG) and N-octyl-β-D-glucopyranoside were purchased from Sigma (St. Louis, Mo., USA). Pioneer Chips L1 were purchased from Biacore (Uppsala, Sweden).

Peptides were synthesised by standard solid phase peptide synthesis methods, using side-chain protected Fmoc-conjugated amino acids in the manual mode. Fmoc-protected amino acids were purchased from Auspep or Novabiochem. The linear sequences were constructed using tritylchloride polystyrene resin (Novabiochem) loaded with the first protected amino acid. The addition of subsequent amino acids was achieved by first deprotecting the Fmoc group followed by coupling of the next amino acid. The cycle was repeated until the last amino acid was coupled, then the Fmoc group was removed with 50% piperidine in dimethylformamide (DMF) and the linear protected peptide was cleaved from the resin with 1% trifluoroacetic acid (TFA) in dichloromethane and collected using pyridine/methanol (3/7) mixture. The linear peptides were then cyclized and purified by HPLC or acetonitrile : water extraction. Table 1 provides an illustration of the peptide sequences of Core peptide (CP), linear sequence of the peptide of formula (I) (C1-L), cyclic C1, cyclic peptide 2 (C2), cyclic peptide 3 (C3) and cyclic peptide 4 (C4).

The cyclized (ring) structures of cyclic peptides C1 to C4 are shown in FIGS. 1 to 4, respectively. Other cyclic peptides are described in Tables 1-3 (below).

### Example 1

### Methods of Cyclization and Purification

Method A (below) is a standard method while Methods B and C (below) have been developed to test for greater yield of product. In Method B cyclization was carried out as per method A but a purification step was introduced before removing protecting groups. Method C uses a different cyclizing reagent as reported by Skropeta et al.

Method A. Cyclization of C1 Using PyBOP

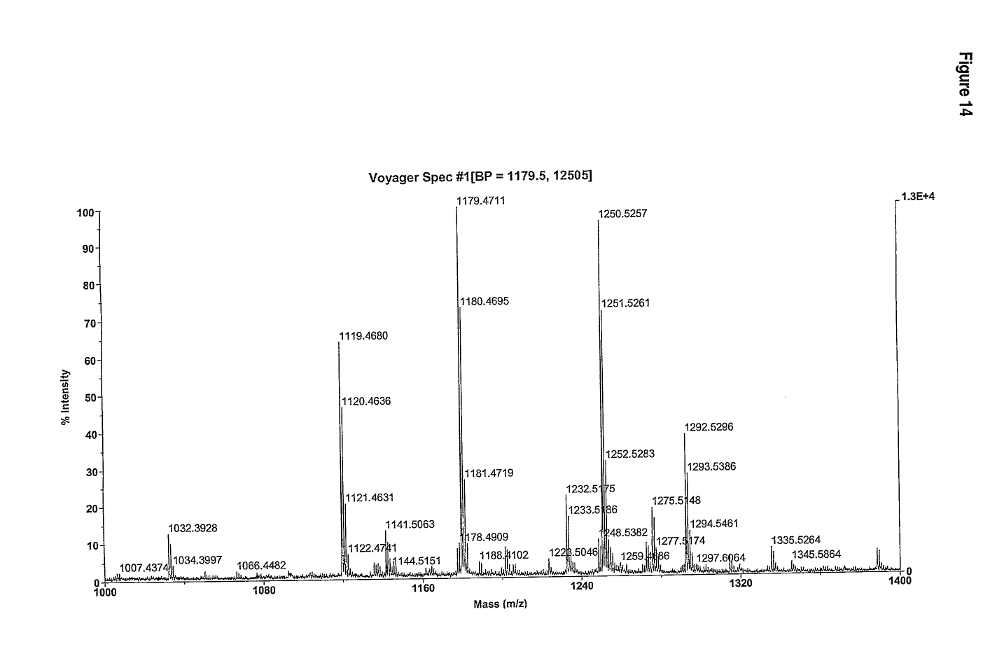

The linear sequence of protected C1 was purchased from Auspep. A solution of the linear peptide in dichloromethane (DCM; ˜1 mM) was stirred with (benzotriazol-1-yloxy)tripyrrolidinophosphonium hexafluorophosphate (PyBOP; 4 eq.) and N,N-diisopropylethylamine (DIEA; 10 eq.) overnight. Cyclized peptide was then dried on rotary evaporator prior to deprotection with a mixture of 95% trifluoroacetic acid (TFA):2.5% water:and 2.5% triisopropylsilane (TIPSI). Crude peptide was characterized by mass spectrometry (FIG. 13).

Method B. Cyclization C1 Using PyBOP and Purification Before Cleavage

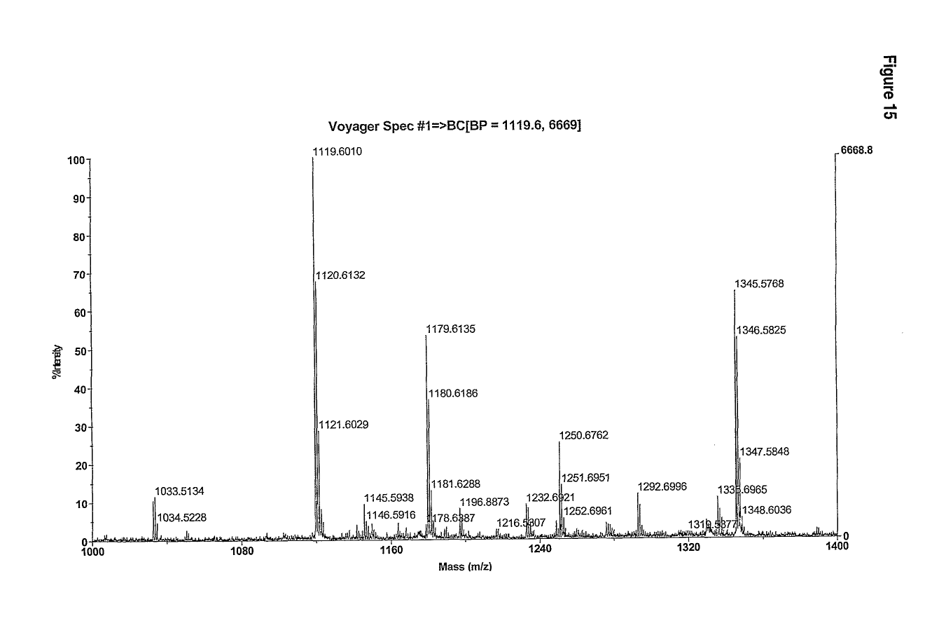

Synthesis was repeated as above except protected cyclized peptide was first precipitated in water and then the precipitate was washed with 0.5M sodium hydrogen carbonate (NaHCO₃), ether and acetonitrile respectively. Protecting groups were then removed as described above in TFA/scavenger mixture and the peptide was characterized by mass spectrometry (FIG. 14).

Method C. Cyclization of C1 Using FDPP

Under anhydrous conditions, 1.5 equiv of pentafluorophenyl diphenylphosphinate (FDPP; 39 mg, 0.1 mmol) was added to a solution of linear protected C1 (100 mg, 0.007 mmol) in acetonitrile (14 ml), followed by addition of 3 equiv of DIEA (0.2 mmol). The reaction mixture was then stirred at room temperature overnight, after which the solvent was removed by rotary evaporation prior to characterization by mass spectrometry (FIG. 15).

HPLC Method for C1 Purification

An At-Column Dilution (ACD) technique developed by WATERS (Waters Applications Note 2003 “At-Column Dilution Application Notes” pp 22 Library No 7150078010, available from www.waters.com) to purify the C1 peptide using HPLC. This technique was developed for injecting relatively large volumes of strong sample diluents. ACD technique also prevents bulk precipitation in the sample loop or in the column itself. FIGS. 16 and 17 below shows the difference between a conventional HPLC system and an ACD system.

C1 was dissolved in approximately 0.5 ml of acetonitrile, 200 μl of 10% TFA and then DMSO was added until a clear solution was obtained. The sample was centrifuged prior to collecting the supernatant for loading on to a C18 reverse phase column. Sample was loaded using ACD techniques. C1 was loaded on to the column at 0.1 ml/min flow rate. Simultaneously mobile phase was pumped through the column using another pump at a flow rate of 5 ml/min. Presence of C1 in fraction collected at a retention time of 31.8-32.2 min was confirmed by mass spectrometry (FIG. 18).

### Example 2

### T-Cell Stimulation in vitro

The cyclic peptides were tested in an antigen presentation assay to examine their effects on T cell stimulation. An established assay (Manolios et al., 1997) was used to assess T cell activation by measuring IL-2 produced in response to antigen. Briefly, 2B4.11 hybridoma (5×10⁴cells) and LK35.2 (5×10⁴) were incubated with pigeon cytochrome C (50 μM) in 96-well microtiter plates for 24 h in the presence and absence of peptides (50, 25, and 10 μM respectively). An aliquot of each supernatant (100 μl) was removed and serial twofold dilutions prepared using medium lacking IL-2. Diluted supernatant was then incubated with CTLL cells (2×10⁴) for 18 h in microtitre plates. [³H] thymidine (0.5 μCi) was added for 6 h and the CTLL cells collected onto glass fibre filter paper using a cell harvester (TiterTekTM). [³H] thymidine uptake was measured by scintillation counting (Hewlett Packard) and compared to standard curves to determine the amount of IL-2 produced. Peptides were initially dissolved in 100% DMSO to concentrations of 10 mM, 5 mM, and 2 mM for storage, and diluted to working concentrations of 50 μM, 25 μM, and 10 μM respectively, all at 0.5% DMSO.

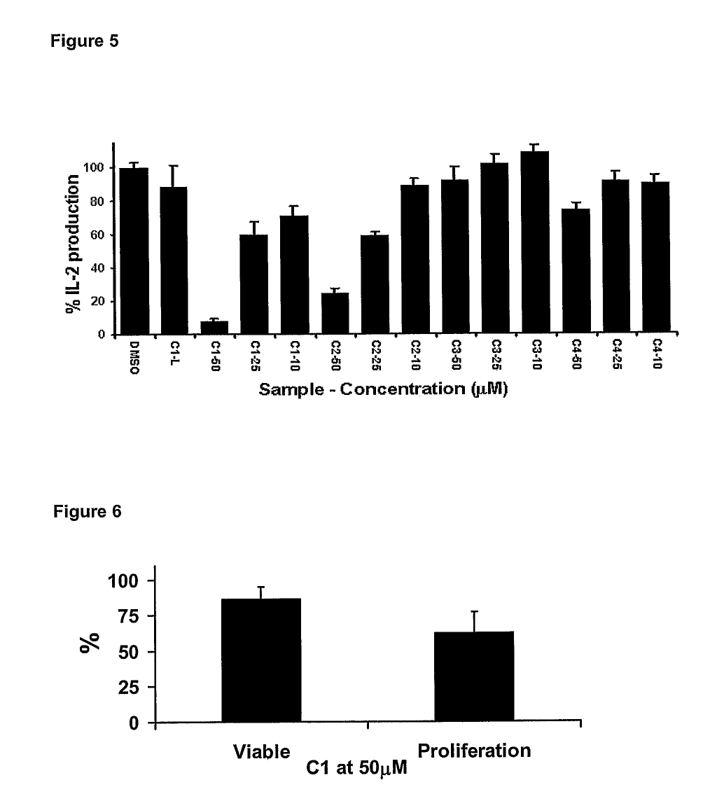

As shown in FIG. 5, each peptide demonstrated dose responsiveness, although only C1 and C2 inhibited IL-2 production appreciably in comparison to a solvent only (DMSO) control. At 50 μM, C1 inhibited IL-2 production to only 7.73%±4.97SD of the solvent only control value, which decreased to 59.69%±23.54SD of the solvent only control value upon lowering the C1 concentration to 25 μM. C2 was the second most active peptide with IL-2 production dropping to 24.51%±8.81SD of the control when tested at 50 μM, and 58.74%±7.63SD IL-2 produced when tested at 25 μM. C3 and C4 showed only minimal activity in this assay at 50 μM, resulting in 91.69%±25.86SD and 74.54%±11.22SD respectively. By comparison CP normally inhibits IL-2 production to 37.08%±10.49SD at equivalent doses (500). Uncyclized C1 (C1-L) also showed only minimal activity at 50 μM.

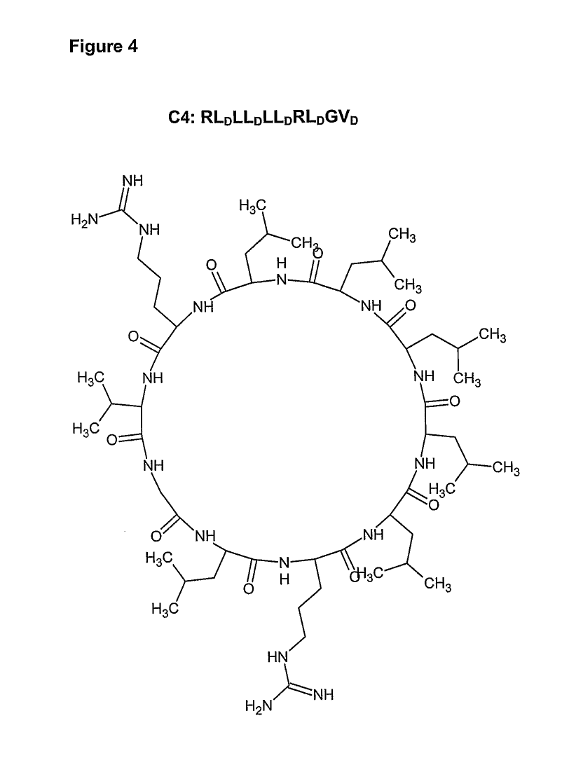

TABLE 1
Peptide list.

| | | % Ave | |
|---|---|---|---|
| | | IL2 Pro- | |
| Sequence | Code | duction | Comment |
| L_{D}RL_{D}LL_{D}LL_{D}KV_{D}G | C1 | 17 | See FIG. 1 |
| L_{D}RL_{D}LL_{D}LL_{D}KV_{D}G | C1-L | 90 | |
| L_{D}LL_{D}LL_{D}LL_{D}LV_{D}G | Cyclic | NT | Gel like proper- |
| | Control | | ties, could not |
| | | | be tested in IL2 |
| | | | assay |
| L_{D}R_{D}L_{D}L_{D}L_{D}L_{D}L_{D}K_{D} | | NT | Small amount |
| V_{D}G | | | cyclized, impure, |
| | | | not tested (NT). |
| LRLLLLLKVG | | NT | Unable to cyclize, |
| | | | NT. |
| L_{D}KL_{D}LL_{D}LL_{D}KV_{D}G | C5 | 78 | Results in Table 4 |
| LR_{D}LL_{D}LL_{D}LK_{D}VG_{D} | C14 | NT | |
| L_{D}LL_{D}RL_{D}LL_{D}KV_{D}G | C6 | 99 | Results in Table 4 |
| R_{D}LL_{D}LL_{D}LL_{D}KV_{D}G | C7 | 49 | Results in Table 4 |
| L_{D}LL_{D}LL_{D}RL_{D}KV_{D}G | C8 | 33 | Results in Table 4 |
| L_{D}RL_{D}KL_{D}LV_{D}LL_{D}G | | NT | Didn't cyclize |
| L_{D}RL_{D}LL_{D}LL_{D}RV_{D}G | C4 | 72 | See FIG. 4 |

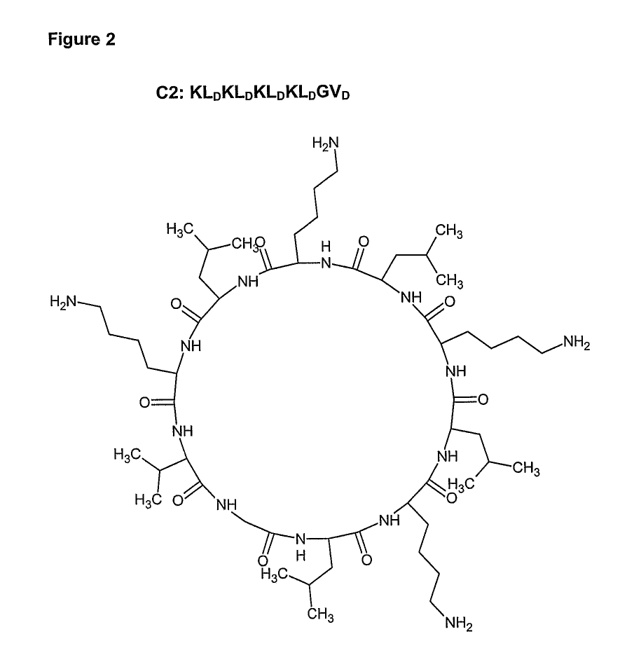

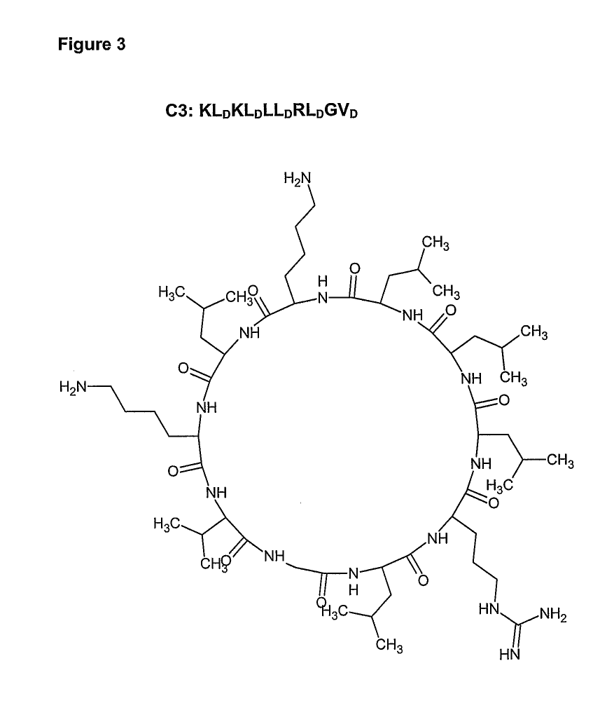

TABLE 2
Cyclic peptides based on C2 and C3

| | | % Ave | |
|---|---|---|---|
| | | IL2 Pro- | |
| Sequence | Code | duction | Comment |
| L_{D}KL_{D}KL_{D}KL_{D}KV_{D}G | C2 | 22 | See FIG. 2 |
| L_{D}RL_{D}LL_{D}KL_{D}KV_{D}G | C3 | 95 | See FIG. 3 |
| L_{D}RL_{D}KL_{D}LL_{D}KV_{D}G | C9 | 25 | Results in (1b) |
| L_{D}RL_{D}KL_{D}KL_{D}KV_{D}G | C10 | 59 | Results in Table 3 |
| L_{D}KL_{D}KK_{D}KL_{D}KV_{D}G | C11 | 96 | Results in Table 3 |
| L_{D}RL_{D}KK_{D}KL_{D}KV_{D}G | C12 | 27 | Results in Table 3 |
| L_{D}KL_{D}KK_{D}KK_{D}KV_{D}G | C13 | NT | Didn't cyclize |
| L_{D}RL_{D}KK_{D}KK_{D}KV_{D}G | | NT | To be synthesized |

TABLE 3
Other sequences

| | | % Ave | |
|---|---|---|---|
| | | IL2 Pro- | |
| Sequence | Code | duction | Comment |
| L_{D}RL_{D}KV_{D}G | 6AA | 35 | Results in |
| | | | Table 3 |
| L_{D}RL_{D}LL_{D}KV_{D}G | 8AA | 72 | |
| GLRILLLKV | All-L-CP | NT | Cyclized but |
| | | | impure, |
| L_{D}SL_{D}RL_{D}LL_{D}LL_{D}KV_{D}G | | NT | |
| L_{D}KL_{D}RL_{D}LL_{D}LL_{D}KV_{D}G | | NT | |

The Examples herein describe that the cyclic peptide C1 has immunomodulatory activity. The Examples demonstrate that related cyclic peptides possess similar such activity and hence offer therapeutic potential.

Based on the permutation studies disclosed herein the inventors have shown that for preferred immunomodulatory activity a cyclic peptide may have an Arginine in position 2 and a Lysine in position 8. Active cyclic peptides may also include an indeterminate number of hydrophobic amino acids between R and K. Furthermore, active cyclic polypeptides with an indeterminate number of charged amino acids between R and K may retain an angle between K and R of 36-140 degrees.

### Example 3

### Activity of Cyclic Peptide C1 in vitro

First, to determine if the activity of C1 was due to toxicity, 2B4.11 T-cells (1×10⁵) were cultured in microtiter wells in the presence of the peptide conjugates at 50 μM. After 20 hours, 30 μL were removed and cells assessed for viability by trypan blue staining. The remaining culture was then pulsed with 0.5 μCi [³H] thymidine for 5 h, harvested, and quantitated for [³H] thymidine uptake to assess cellular proliferation.

As shown in FIG. 2, C1 at 50 μM caused a decrease in cellular proliferation to 62.56%±14.25SD whereas cell viability decreased to 86.63%±8.14SD. Thus, the immunmodulatory effect of C1 does not appear to be due to toxicity caused by C1.

T-Cell Activation

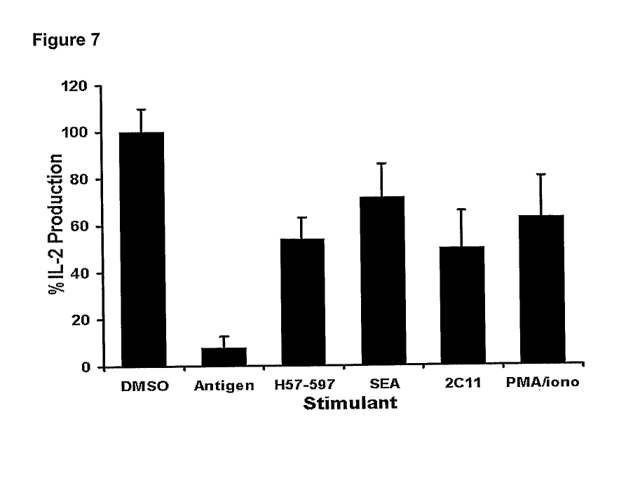

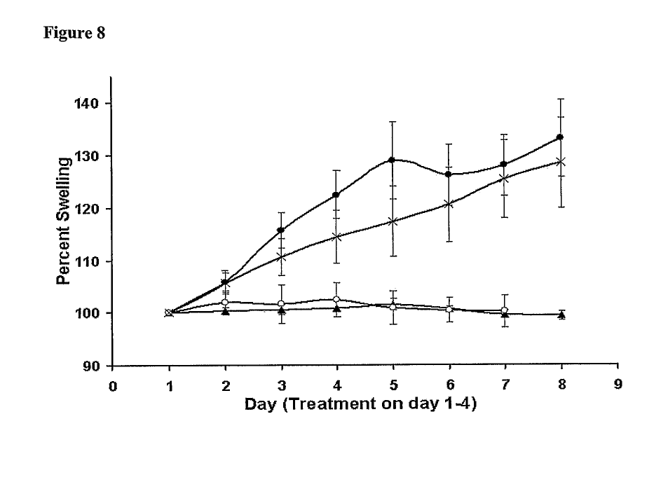

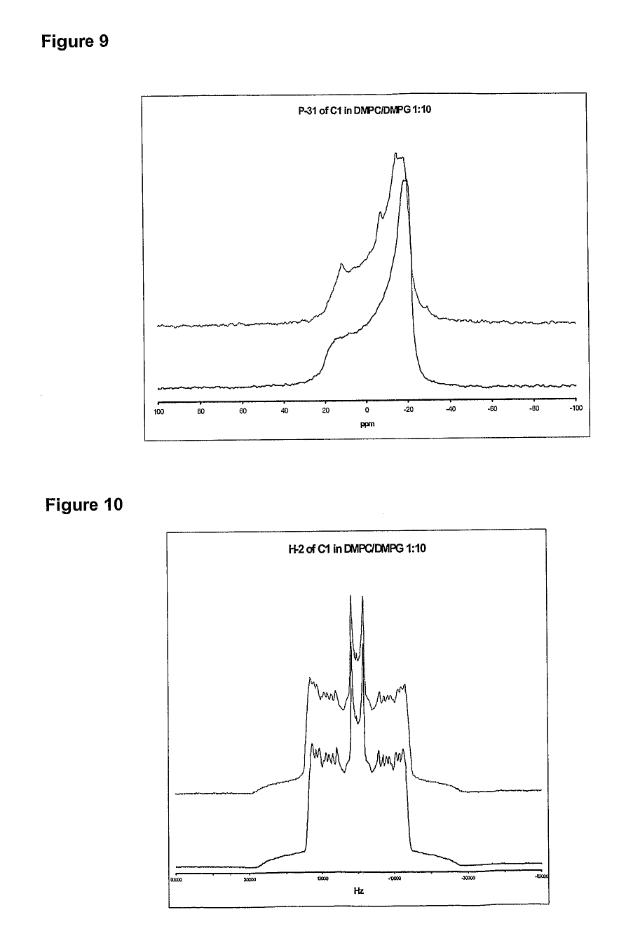

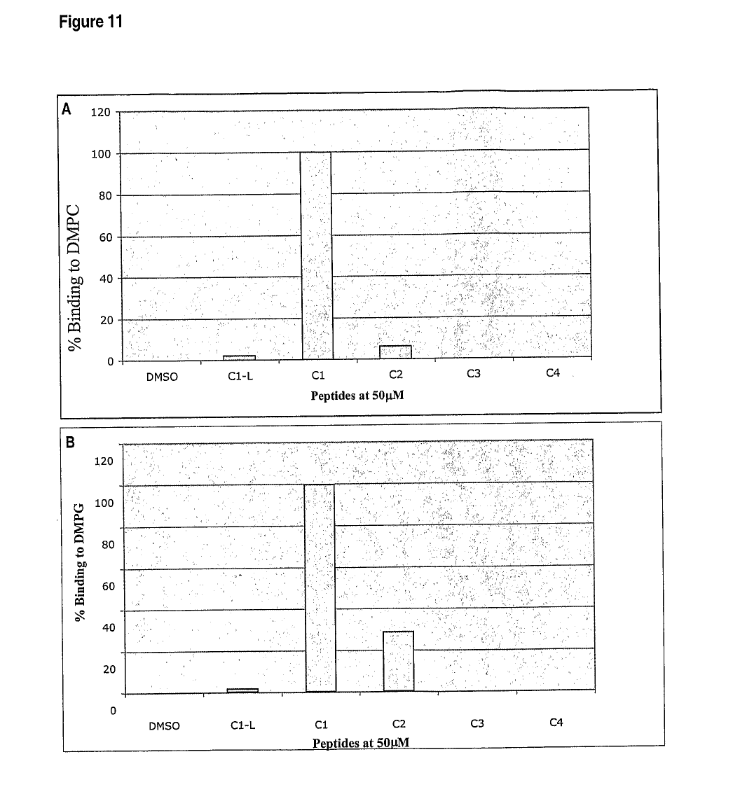

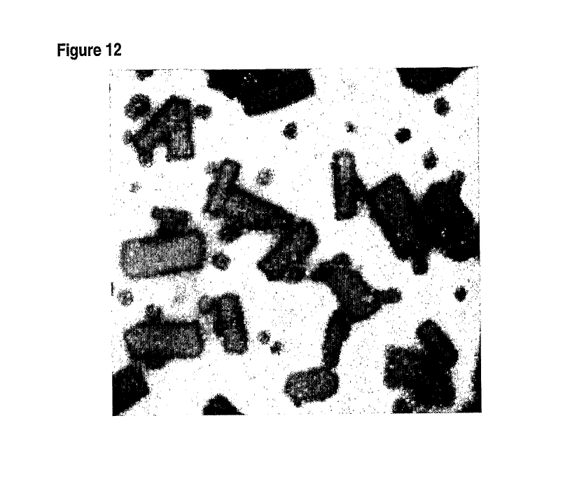

To analyse the mode of action of C1, T-cells were activated using 2C11-145 and H57-597 antibodies. The H57-597 antibody is directed against the TCRβ chain, and when administered at sub-optimal doses in conjunction with accessory cells (LK35.2), is capable of activating T cells. T-cell hybridoma, 2B4.11 cells (1×10⁵) were cultured in 96-well, round bottom plates in the presence of LK cells (5×10⁴), 0.5 μg/mL of either soluble 145-2C11 (lab purified) to crosslink the CD3ε chains of the TCR, or H57-597 (Biolab) to crosslink the TCRβ chain. C1 was added to give a final reaction volume of 250 μL at 50 μM and 0.5% DMSO. Plates were incubated for 24 h at 37° C. with 5% CO₂. Supernatants were then collected and analysed for IL-2 content. All conjugates were tested simultaneously in triplicate wells, and each experiment was performed on three separate occasions. C1 inhibited IL-2 production to 54.02%+/−9.32SD using H57-597 (see FIG. 7). The results for other peptides are shown in Table 4.

T cells were also activated using the 2C11-145 antibody to crosslink the CD3 chains of the TCR, thereby bypassing the antigen recognition sub-units of the TCRα/β. CD3 crosslinking in the presence of C1 resulted in a % IL-2 production of 50.00%+/−15.79SD (see FIG. 7).

T-cells were also activated using Staphylococcal enterotoxin A (SEA). SEA is capable of activating T-cells through the TCR. In the present system however, the MHC molecule of the macrophage (LK35.2) is crosslinked to the TCR β subunit resulting in T-cell activation. The T-cell hybridoma, 284.11 (5×10⁴ cells) were incubated with an equal amount of the macrophage, LK35.2 (5×10⁴ cells), at 100 ng/mL of SEA, 50 μM of C1, and 0.5% DMSO to a total volume 250 μL. Stimulation was allowed to proceed for 24 hours at 37° C./5% CO₂ in 96-well in round bottom microtiter plates. Supernatants were then collected and analysed for IL-2 production. C1 inhibited IL-2 production to 71.42%+/−14.275D using the SEA model of T-cell activation (see FIG. 7). The results for other peptides are shown in Table 4.

Further, T-cells were activated using PMA and ionomycin stimulation. The method employed to activate T cells utilized the protein kinase C activating PMA in combination with the calcium ionophore, ionomycin. In combination, PMA/ionomycin effectively activate T cells downstream from the TCR. Phorbol 12-myristate 13-acetate (PMA) (40 ng/mL) and ionomycin (5 μg/mL) were added to 2B4 cells (5×10⁴) and incubated with each CP-conjugate at 50 μM in 0.5% DMSO in 96-well, round bottom microtiter plates for 24 h at 37° C. with 5% CO₂ in a total volume of 250 μL. Supernatants were then collected and analysed for IL-2 production. All CP-conjugates were tested simultaneously in triplicate wells, and each experiment was performed on three separate occasions. PMA/ionomycin activation in the presence of C1 resulted in an IL-2 production of 62.83%+/−17.60SD relative to the DMSO control (see FIG. 7). The results for other peptides are shown in Table 4.

The above observations, when considered together with the cell viability and cell proliferation results, indicate that C1 is a very sensitive and potent inhibitor of T-cell activation under the normal antigen presenting system requiring the “macrophage” presenting the antigen to the T-cell of interest. Of all the activation systems tested, the antigen presentation mechanism is the closest representation of what actually occurs in an in-vivo environment. In this light, the fact that C1 inhibits IL-2 production demonstrates its potential for use as a treatment option for T-cell mediated conditions.

In Table 4 (below) each IL-2 column represents individual experiments performed in triplicate. The values show the average amount of IL-2 produced (%) relative to DMSO control (100%). That is, the smaller the values the more inhibition (effectiveness).

TABLE 4
Results of antigen Presenting Assay of new cyclic peptides

| | | IL2 | IL2 | IL2 | IL2 | IL2 |
|---|---|---|---|---|---|---|
| Peptide | Sequence | % | % | % | % | % |
| CP (linear) | G L R I L L L K V | 20.9 | 25 | 20.9 | 13.5 | 17.4 |
| C1 (cyclic) | L_{D} R L_{D} L L_{D} L L_{D} K V_{D} G | — | — | — | 18.9 | 15.6 |
| C5 (cyclic) | L_{D} K L_{D} L L_{D} L L_{D} K V_{D} G | 73.6 | 75 | 85.4 | — | — |
| C6 (cyclic) | L_{D} L L_{D} R L_{D} L L_{D} K V_{D} G | 104.5 | 104 | 89 | — | — |
| C7 (cyclic) | R_{D} L L_{D} L L_{D} L L_{D} K V_{D} G | 29 | 15.4 | 54 | 71 | 76.4 |
| C8 (cyclic) | L_{D} L L_{D} L L_{D} R L_{D} K V_{D} G | — | 35 | (61.2) | 20.2 | 17.4 |
| C9 (cyclic) | L_{D} R L_{D} K L_{D} L L_{D} K V_{D} G | — | 11.5 | (65.3) | 6.75 | 16.9 |
| C10 (cyclic) | L_{D} R L_{D} K L_{D} K L_{D} K V_{D} G | — | — | — | 54 | 64.1 |
| C11 (cyclic) | L_{D} K L_{D} K K_{D} K L_{D} K V_{D} G | — | 88.5 | 104 | — | — |
| C12 (cyclic) | L_{D} R L_{D} K K_{D} K L_{D} K V_{D} G | 9.0 | 21 | (49) | 33.7 | 24 |
| 6AA (cyclic) | L_{D} R L_{D} K V_{D} G | 9.0 | 36.5 | (52) | 33.7 | 44.3 |
| Cyclosporin A | — | — | nd | nd | — | — |

Peptide concentration, 50 μM, not detected

### Example 4

### Activity of Cyclic Peptide C1 in vivo

The immunomodulatory effects of C1 in vivo were examined by using an adjuvant-induced arthritis model in Wistar rats. Arthritis in rats was induced by a single subcutaneous injection of 1 mg heat killed Mycobacterium tuberculosis [MTB] in 100 μL squalane (adjuvant) at the base of the tail as previously described (Manolios et al. 1997). Rats generally develop is arthritis 12-14 days after the injection. At the onset of arthritis rats were injected with four daily injections of C1 (6 mg) and the effect on arthritis assessed. At regular intervals between days 0-28, animals were weighed and their arthritic condition assessed by measurement of weight, hind paw thickness, hind paw width, hind ankle thickness (with a micrometer screw gauge) and the number of arthritic joints was recorded.

The effectiveness of C1 in vivo was compared to commercially available cyclosporin. C1 given subcutaneously had a comparable effect to cyclosporin in reducing inflammation in the adjuvant induced arthritis model. This was more significantly favourable when compared with CP or placebo treated rats. The difference in swelling observed between C1 treated rats and those treated with the placebo was statistically significant (P<0.0001), whereas the difference between C1 and cyclosporine treated rats was not statistically significant.

### Example 5

### Biophysical Studies Using Cyclic Peptide C1

Solid-State NMR Spectroscopy

Samples containing 25 μmol of lipid (either DMPC-d₅₄ alone or DMPG/DMPC-d₅₄ at a 1:3 molar ratio) were prepared by dissolving the dry lipids in 500 μL of methanol/chloroform (1/1 v/v), followed by addition of the appropriate amount of peptide in 500 μL of trifluoroethanol. Subsequently, the solvents were removed by drying in a rotary evaporator followed by overnight incubation under high vacuum. The peptide/lipid mixed films obtained were hydrated with 150 μL of distilled water, and the resulting dispersions of multilamellar vesicles were subjected to 10 freeze-thaw cycles to ensure that the samples were homogeneous.

NMR spectra of the peptide-lipid dispersions were recorded at 34° C. on a Varian (Palo Alto, Calif.) Inova 300 wide-bore NMR spectrometer, using a broadband probe with a 5 mm sample coil. Proton-decoupled ³¹P experiments were carried out at 121.46 MHz (4 μs 90° pulse, 1.5 s recycle time. Approximately 10,000 scans were acquired using a sweep width of 25 kHz and 1024 complex data points. Prior to Fourier transformation an exponential multiplication was applied, resulting in a 100 Hz line broadening. ²H NMR measurements were performed at 46.06 MHz, using a quadrupolar echo sequence (Davis et al., 1976) (8 μs 90° pulse, 30 μs pulse separation), a recycle time of 0.5 s, and a spectral width of 100 kHz, with 4000 complex data points in the time domain. Approximately 60,000 scans were accumulated. The free induction decays were left-shifted to begin at the top of the echo and multiplied with an exponential window function equivalent to a line broadening of 50 Hz. All spectra were scaled to the same height.

³¹P and ²H NMR measurements were performed to investigate whether the C1 peptide is capable of perturbing model membrane structure. A measurement temperature of 34° C. was used to ensure that the model membranes were in the biologically relevant liquid crystalline (L_{α}) phase. ³¹P NMR spectra, which give information about the organization and dynamics of the lipid headgroups (Seelig, 1978) are shown in FIG. 5 for dispersions of DMPC-d₅₄/DMPG (1:10) in the absence and presence of C1.

The conformation of the lipid acyl chains were then assessed by ²H NMR measurements on membranes that contain lipids with perdeuterated acyl chains. The ²H NMR spectrum of a dispersion of DMPC-d₅₄ /DMPG in the absence and presence of peptide is presented in FIG. 6. The spectrum consists mostly of overlapping doublet resonances that resulted from the different CD₂ segments of the acyl chain, while the central high-intensity doublet corresponds to the terminal CD₃ moiety. The above solid state NMR studies of C1 at 10% concentration in DMPC/DMPG membranes show that the peptide appeared to order the lipid suggesting a io transmembrane orientation. Based on these observations, although C1 appears to enter membranes and adopt a transmembrane orientation, it does not appear likely that it is forming lethal pores.

Surface Plasmon Resonance (SPR)

SPR is a useful method in the study of peptide binding and possible insertion into membranes. Successful applications include studies of antimicrobial peptides and their mode of action such as melittin, its analogues and magainin and to understand the mode of action of membrane-active peptides using anionic and zwitterionic lipids. The technique of SPR offers the ability to study structure/function relationships while overcoming the need to use labelled peptides as well as cumbersome separation procedures (see Salamon et al., 1999). It allows real time monitoring of peptide binding to, and dissociation from, these model membranes.

Model membranes composed of zwitterionic and anionic phospholipids were prepared as previously described (Bender et al., 2004). Briefly, DMPC and DMPG were dissolved in dry chloroform and chloroform/methanol (2:1) respectively to give 10 mg/ml solutions. These were evaporated under reduced pressure and the resulting lipid films dried overnight in vacuo. Lipids were hydrated by resuspending in N-2-hydroxyethylpiperazine-N′-2-ethanesulfonic acid (HEPES) buffer for 60 min at 34° C. to give 0.5 mM concentration in respect of phospholipids. The solution was sonicated in an ultrasonic bath for 20 min. Eight cycles of freeze/thawing was followed by extrusion through polycarbonate filters, first 100 nm (21 times) then 50 nm pore diameter (21 times), using a Lipofast apparatus (Avestin, Ottawa, Canada) and SUVs were used immediately.

SPR was carried out on a BIAcore 2000 instrument using Pioneer Sensor Chip L1 and HEPES (HBS-N, Biacore, Upssala, Sweden) as running buffer. The chip surface was cleaned with 40 mM octyl glycoside (30 μl, 10 μl/min) followed liposomes (SUV) (100 μl, 5 μl/min). 10 mM NaOH (40 μl, 10 μl/min) removed any multilamellar vesicles from the surface before injecting the lipopeptide solutions (100 μl, 5 μl/min). The dissociation stage was 1200 sec. Regeneration of the sensor chip was achieved with 40 mM octyl glycoside (30 μl, 10 μl/min). All SPR experiments were run at 25° C. and all analyses were performed using the BIAevaluation software (Biacore, Uppsala, Sweden). Lipopeptide binding was expressed as the difference in signal between points before sample injection and after dissociation.

In the SPR studies conducted, binding of cyclic peptides to DMPC and DMPG liposomes was investigated. It was found that C1 bound much stronger to both model membranes than any of the other peptides tested (FIG. 7). Binding affinity of C1 to DMPC model membrane is approximately 10 fold higher than CP. The binding was irreversible and the compounds could not be removed from the membrane surface even after prolonged washing with elution buffer. The analogues C3 and C4 showed no binding at all to either of the model membranes and the uncyclized C1 (C1-L) only very limited binding. Sensorgrams of C2 showed that there was some binding to the anionic liposome (3000 RU) but only very limited binding to the zwitterionic (data not shown). With the exception of C2, C1 demonstrated in excess of 10-fold binding to all the other analogues examined. The inventors have recently reported that there is a strong correlation between the membrane binding affinity and functional activity of CP and its linear analogues in inhibiting IL-2 production (Bender et al., 2004). Here it is shown that this correlation also holds true for C1. C1 has a higher binding affinity to model membranes than that of CP and also exhibits a stronger biological activity.

Transmission Electron Microscopy Method

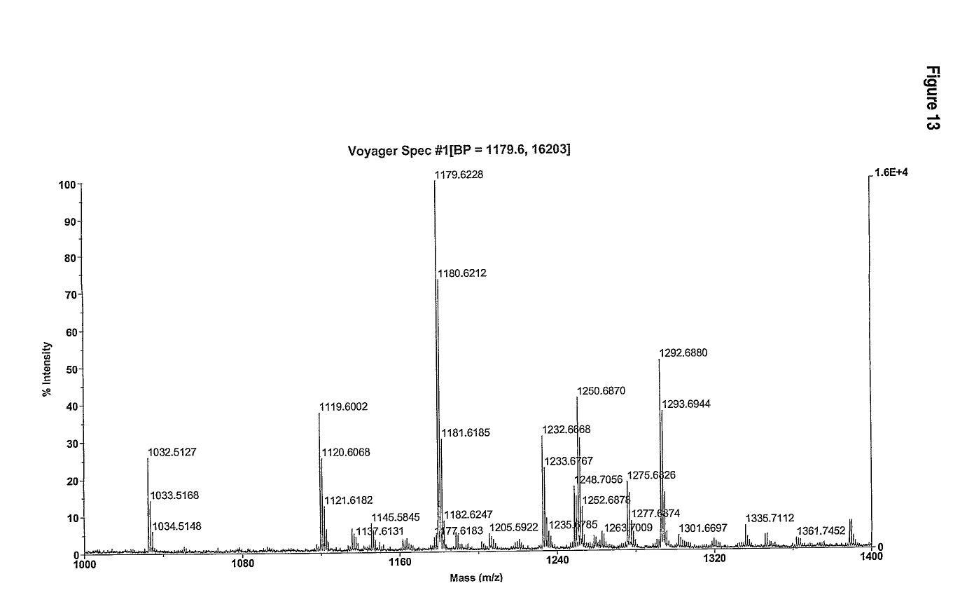

Transmission electron microscopy was used to identify if C1 formed nanotubes. A suspension of C1 in water, 90%acetonitrile: 10% water, methanol, water/DMSO and water/HCl were prepared. These suspensions were made at a range of concentrations (6 μM-2 mM). None of the suspensions, except for water/HCl indicated any tube like structures shown in FIG. 12. Further analysis is currently underway to assess if these tube-like structures are nanotubes.

C1 in water/HCl sample was prepared as follows: C1 (1.5 mg) was suspended in 100 μl of water and 50 μl of 10 mM HCl to make an approximately 9 mM stock solution. The stock solution was then diluted to a final concentration of 100 μM using water. Then a small drop of C1 was applied to Pioloform coated 400 mesh Cu electron microscope grids for one minute. Excess liquid was removed by blotting before floating the grid for one minute in 1% phosphotungstic acid stain and then blotting the excess liquid. Grids were then mounted in a single tilt specimen holder and examined in a Philips (Eindhoven, The Netherlands) CM120 Biofilter Transmission Electron Microscope operated at 120 kV. Digital images were captured using a Gatan (Pleasanton, USA) Model 690 Slow-scan CCD camera.

### Example 6

### Enzyme and pH Stability of Cyclic Peptide C1

The effect of trypsin on C1 and CP was studied by incubating each peptide (1 μg/μl) in methanol and ammonium bicarbonate (0.1M) with 125 ng/μl of trypsin at pH 8 and 37° C. The effect of trypsin after incubation with CP and C1 was followed by mass spectrometry. As s expected CP was cleaved by trypsin producing degradation product ILLLK, while C1 stayed intact after incubation for the same length of time. The results are illustrated in Table 2 below.

pH stability was also investigated. C1 (2 μg/μl) was incubated at 30° C. for three days at pH 3, 7 and 10. Samples were taken after each day and analysed by mass spectrometry. C1 did not degrade after incubation at different pH environments (Table 5).

TABLE 5
Enzyme and pH stability of C1 and CP

| | 3 hours (Mass | |
|---|---|---|
| Sample | spectroscopy-MS) | 24 hours (MS) |
| C1 and trypsin | Peak at 842 due to | Peak at 842 due to |
| | trypsin cleaving | trypsin cleaving |
| | itself but no | itself but no |
| | degradation products | degradation products |
| | from C1 | from C1 |
| C1 without trypsin | No degradation product | No degradation product |
| CP and trypsin | Peak at 599 (ILLLK) | Peak at 599 (ILLLK) |
| | degradation product | degradation product |
| CP without trypsin | No degradation product | No degradation product |

### Example 7

### Antimicrobial Activity of Cyclic Peptide C1

The synthetic C1 peptide and its analogues were tested using Standard disk susceptibility testing by National Committee for Calibration Laboratory Standards (NCCLS) against, S. aureus, E. coli and S. pneumoniae. From all the cyclic peptides tested only C1 showed antimicrobial activity against S. aureus and the MIC was determined to be 100 μg/ml. The results are illustrated in Table 6.

TABLE 6
Antimicrobial activity of C1 and analogues

| | | | |
|---|---|---|---|
| Peptide (μg/ml) | S. aureus | E. coli | S. pneumoniae |

| | | | | |
|---|---|---|---|---|
| C1 | (1000) | +ve | −ve | −ve |
| C1 | (100) | +ve | −ve | −ve |
| C1 | (10) | −ve | −ve | −ve |
| C2 | (1000) | −ve | −ve | −ve |
| C2 | (100) | −ve | −ve | −ve |
| C2 | (10) | −ve | −ve | −ve |
| C3 | (1000) | −ve | −ve | −ve |
| C3 | (100) | −ve | −ve | −ve |
| C4 | (1000) | −ve | −ve | −ve |
| C4 | (100) | −ve | −ve | −ve |
| C4 | (10) | −ve | −ve | −ve |
| Oxacillin | (1) | +ve | −ve | +ve |
| Ceforaxime | (30) | −ve | +ve | −ve |

### Example 8

### Dose and Administration Route of C1

The effect of administering an immunosuppressive peptide on the development of allergic airways disease in a mouse model was determined using female Balb/c WT mice at 7 weeks old housed in IVC cages. As a 400 μg intraperitoneal dose of cyclic C1 peptide results in death of mice 50, 100 or 200 μg doses of cyclic C1 were administered to groups of mice either subcutaneously or intraperitoneally according to the design set out below.

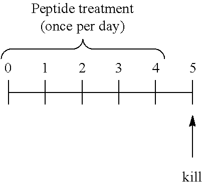

| | | | Dose | | | | |
|---|---|---|---|---|---|---|---|
| | | | (μg/ | | | | |
| | # | | 100 | | | | End- |
| Group | mice | Peptide | μl) | Route | Day 0 | Day 5 | points |
| 1 | 4 | Cyclic | 50 | Intra- | 23/09/09 | 28/09/09 | none |
| | | | | peri- | | | |
| | | | | toneal | | | |
| 2 | 4 | Cyclic | 100 | Intra- | 23/09/09 | 28/09/09 | none |
| | | | | peri- | | | |
| | | | | toneal | | | |
| 3 | 4 | Cyclic | 200 | Intra- | 23/09/09 | 28/09/09 | none |
| | | | | peri- | | | |
| | | | | toneal | | | |
| 4 | 4 | Cyclic | 50 | Subcu- | 23/09/09 | 28/09/09 | none |
| | | | | taneous | | | |
| 5 | 4 | Cyclic | 100 | Subcu- | 23/09/09 | 28/09/09 | none |
| | | | | taneous | | | |
| 6 | 4 | Cyclic | 200 | Subcu- | 23/09/09 | 28/09/09 | none |
| | | | | taneous | | | |

### Observations

Day 0: Following the initial cyclic C1 peptide intraperitoneal injection mice were continuously monitored. Mice in group 3 were lying on the bottom of the cage within 10-15 minutes and their breathing rate had slowed. These animals were not moving around or grooming as mice usually do. The eyes of a few of these mice also appeared darker. In group 3, one mouse's tail began to appear blue and this animal was euthanased immediately and the remainder of the group were also euthanased (total of 4 mice) as by this stage most had begun to exhibit signs of distress (approximately 1 hour and 15 minutes after the initial injection). Mice in groups 1 & 2 appeared slightly distressed (scruffy and hunched) for the first 60 minutes however showed no further signs of distress were observed such as those observed with group 3. By 1 hour and 15 minutes after injection the mice in groups 1 and 2 mice appeared to be improving (beginning to move around the cage) and by 2 hours were behaving as normal. Mice in groups 4-6 did not show any adverse effects except for some minor irritation (scratching) at the site of injection.

Day 1: Mice in groups 1 & 2 appeared slightly scruffy and hunched following the injection but fully recovered by 1 hour. Mice in groups 4-6 did not show any adverse effects.

Day 2: Mice in groups 1 & 2 appeared slightly scruffy and hunched following the injection but fully recovered by 1 hour. Mice in groups 4-6 did not show any adverse effects.

Day 3: All mice appeared normal with no sign of adverse effects.

Day 4: All mice appeared normal with no sign of adverse effects.

Day 5: All mice sacrificed.

### Conclusion

Mice did not tolerate 200 μg cyclic C1 delivered by intraperitoneal injection. A 200 μg dose of cyclic C1 delivered by subcutaneous injection did not cause any adverse effects and was used in further studies (see below).

### Example 9

### Subcutaneous Administration of C1 in OVA Model

The effect on the inflammatory response of the 200 μg dose and subcutaneous route of administration of cyclic C1 peptide determined in Example 8 was tested with the OVA (ovalbumin) model (Temelkovski, et al. (1998); Foster, et al. (2000); Asquith et al (2008)) according to the following study design.

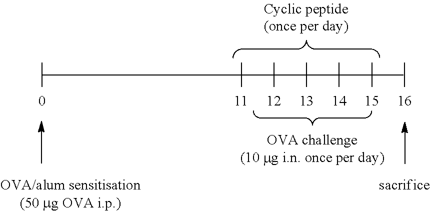

| | | | Dose | | | | |
|---|---|---|---|---|---|---|---|
| | | | (μg/ | | | | |
| | # | | 100 | | | | End- |
| Group | mice | Peptide | μl) | Route | Day 0 | Day 16 | points |
| 1 | 4 | Cyclic | 200 | Subcu- | 26/09/09 | 12/10/09 | none |
| | | | | taneous | | | |

### Inflammatory Cells in Bronchoalveolar Lavage Fluid (BALF)

At the endpoint mice were euthanased and BALF was obtained by cannulating the trachea and flushing the airways with two 0.8 ml volumes of Hank's Balanced Salt Solution (HBSS). Recovered cells were treated with erythrocyte lysis buffer, cytospins prepared and stained with May-Grunwald Giemsa and differential leukocyte counts performed based on morphological criteria (min 200 cells counted/slide). Statistical analysis was performed using a student's unpaired t-test. Results were compared to historical data.

### Results

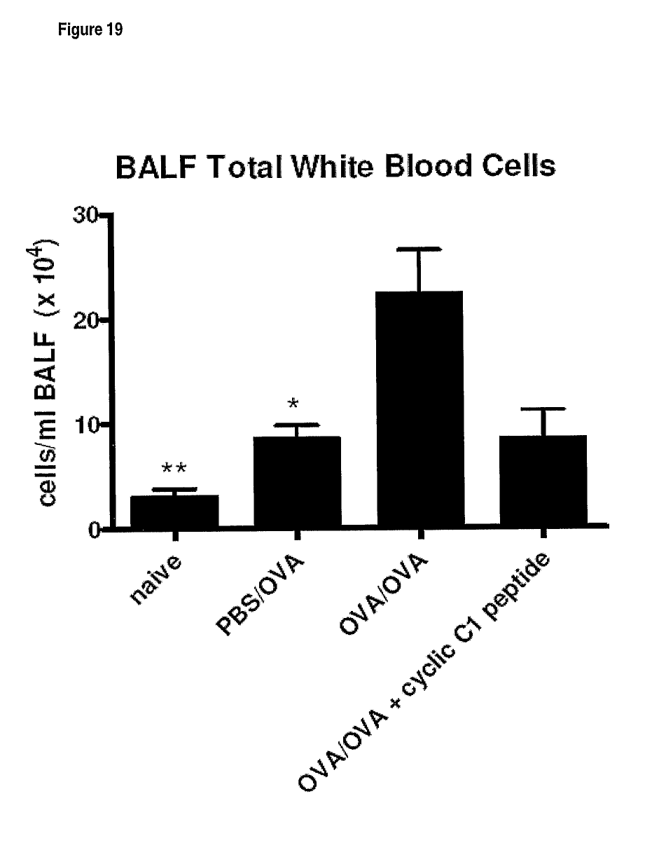

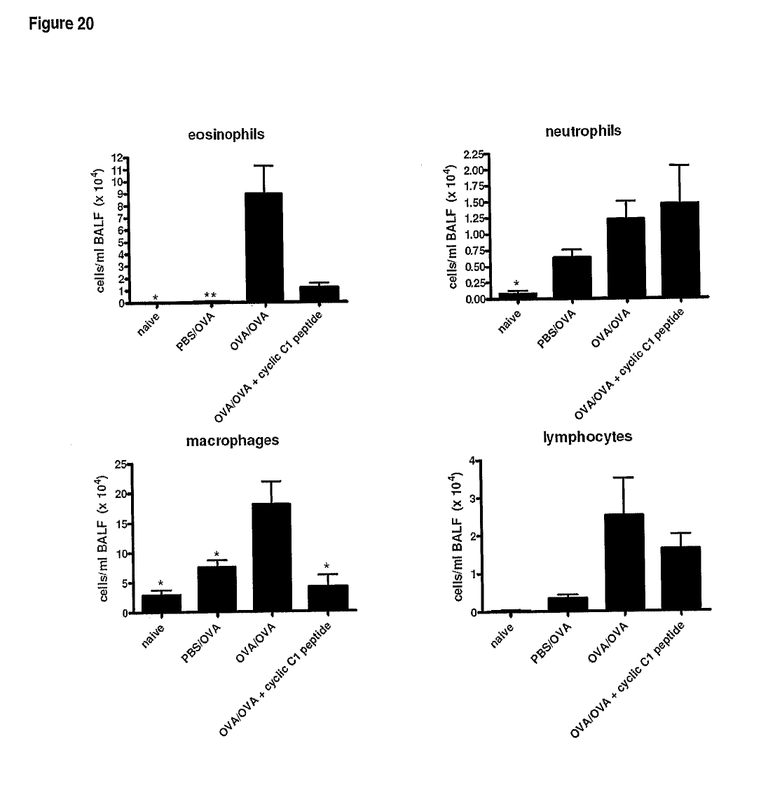

Administration of 200 μg of the cyclic C1 peptide via subcutaneous injection in combination with the OVA model did not cause any adverse reactions in the mice except for some minor irritation (scratching) at the site of injection. Cyclic C1 peptide suppressed the immune response as indicated by a reduction in total inflammatory cells (FIG. 19). This reduction in inflammatory cells is associated with a decrease in eosinophils and significant reduction in macrophages (FIG. 20).

### Example 10

### Immunosuppressive Peptides in Allergic Airway Disease

The effect of administration of immunosuppressive peptides during the challenge phase of a mouse model of allergic airways disease was determined by administration of cyclic C1 peptide with the OVA (ovalbumin) model according to the following study design.

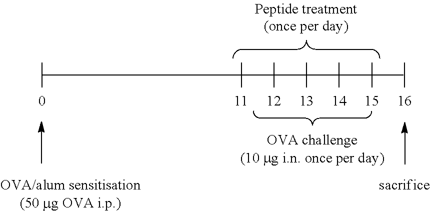

| | | | | |
|---|---|---|---|---|
| | | | | Peptide |
| | | | | treatment |
| | | | | during |
| Group | AAD treatment | sensitisation | challenge | challenge |

Groups:

| | | | | |
|---|---|---|---|---|
| A | PBS/OVA | PBS/alum i.p. | 10 μg OVA | None |
| | | | i.n. | |
| B | OVA/OVA | 50 μg OVA/ | 10 μg OVA | None |
| | | alum i.p. | i.n. | |
| C | OVA/OVA + | 50 μg OVA/ | 10 μg OVA | Linear C1 |
| | linear C1 | alum i.p. | i.n. | peptide |
| | peptide | | | (200 μg/ |
| | | | | 100 μl s.c.) |
| D | OVA/OVA + | 50 μg OVA/ | 10 μg OVA | Cyclic C1 |
| | cyclic C1 | alum i.p. | i.n. | peptide |
| | peptide | | | (200 μg/ |
| | | | | 100 μl s.c.) |
| E | OVA/OVA + | 50 μg OVA/ | 10 μg OVA | Core peptide |
| | core peptide | alum i.p. | i.n. | (200 μg/ |
| | | | | 100 μl s.c.) |
| F | OVA/OVA + | 50 μg OVA/ | 10 μg OVA | Methyl |
| | Methyl | alum i.p. | i.n. | prednisolone |
| | prednisolone | | | (200 μg/ |
| | | | | 100 μl s.c.) |

The endpoints of this study include Bronchoalveolar lavage inflammatory infiltrates, Local (peribronchial lymph node) and systemic (spleen) antigen specific cytokine production (IL-5 and IL-13) and Airway hyperreactivity.

### Inflammatory Cells in Bronchoalveolar Lavage Fluid (BALF)

BALF was obtained by cannulating the trachea and flushing the airways with two 0.8 ml volumes of Hank's Balanced Salt Solution (HBSS). Recovered cells were treated with erythrocyte lysis buffer, cytospins prepared and stained with May-Grunwald Giemsa and differential leukocyte counts performed based on morphological criteria (min 200 cells counted/slide). Statistical analysis was performed using a student's unpaired t-test.

### Local and Systemic Cytokine Production

Peribronchial lymph nodes (PBLN) and spleens were filtered through 70 μm nylon mesh, treated with erythrocyte lysis buffer and cultured at 37° C./5% CO₂ in 96-well plates at 1×10⁶ cells/well in animal cell culture medium (ACCM; 0.1 mM sodium pyruvate, 2 mM L-glutamine, 20 mM HEPES, 100 U/ml penicillin/streptomycin, 50 μM 2-ME and 10% FBS in RPMI1640). Cells were either unstimulated (media only), or antigen-stimulated (200 μg/ml OVA). Cultures were incubated for 5 days and supernatants stored at −70 ° C. until analysis. IL-5 (BD Pharmingen) and IL-13 (R&D Systems) concentrations were determined in cell free supernatants by ELISA according to the supplier's recommendations. Statistical analysis was performed using a student's unpaired t-test.

### Measurement of Airways Hyperreactivity (AHR)

Airway hyperreactivity to inhaled-methacholine representative of the large (Transpulmonary resistance, RL) and small (dynamic compliance, Cdyn) airways was determined. Animals were anesthetized by intraperitoneal injection of ketamine-xylazine and tracheostomised with insertion of a polyethylene cannula (i.d. 0.813 mm). The tracheal tube was connected to a ventilation port within the plethysmograph chamber, and this port was connected to a rodent ventilator (HSE Minivent Type 845, Hugo Sachs Elektronik, Harvard, Germany). Mice were mechanically ventilated at a rate of 170 breaths per minute with a stroke volume of 175 μl. Volume changes due to thoracic expansion with ventilation were measured by a transducer connected to the plethysmograph flow chamber. A pressure transducer measured alterations in tracheal pressure as a function of airway caliber. Once stabilized, mice were challenged with saline, followed by increasing concentrations of methacholine (0.625, 1.25, 2.5, 5.0 and 10.0 mg/ml). Aerosols were generated with an ultrasonic nebuliser (Buxco, Aeroneb Laboratory Nebulizer) and delivered to the inspiratory line. Each aerosol was delivered for a period of 5 minutes, during which pressure and flow data were continuously recorded, and specialist software (BioSystemXA, Buxco Electronics, Inc.) was used to calculate pulmonary resistance and compliance. Peak values were taken as the maximum response to the concentration of methacholine being tested, and were expressed as the percentage change over the saline control. Statistical analysis was performed using a two-way ANOVA.

### Example 11

### Conclusions

Table 7 is a summary of the effect of peptide administration on a spectrum of parameters associated with allergic airway disease. Mice were treated with linear C1 peptide, cyclic C1 peptide, core peptide or methylprednisolone subcutaneously during OVA challenge. Statistically significant differences compared to mice given OVA/OVA alone (‘allergic mice’) are summarised.

TABLE 7
Summary of the effect of C1 Administration

| | | | O/O + | O/O + |
|---|---|---|---|---|
| | O/O + linear | O/O + cyclic | core | methylpred- |
| | C1 peptide | C1 peptide | peptide | nisolone |
| Airway | ↓ | ↓ | ↓ | ↓ |
| inflammatory cells | | | | |
| Airway neutrophils | nc | nc | ↓ | ↓ |
| Airway | nc | nc | nc | nc |
| lymphocytes | | | | |
| Airway eosinophils | ↓ | ↓ | ↓ | ↓ |
| Airway | nc | nc | nc | ↓ |
| macrophages | | | | |
| Airway Resistance | nc | nc | ↓ | ↓ |
| Airway | nc | ↑ | ↑ | ↑ |
| Compliance | | | | |
| Spleen OVA- | ↓ | ↓ | ↓ | ↓ |
| stimulated IL-5 | | | | |
| Spleen OVA- | nc | ↓ | ↓ | ↓ |
| stimulated IL-13 | | | | |
| LN OVA- | nc | ↓ | ↓ | nc |
| stimulated IL-5 | | | | |
| LNOVA | nc | ↓ | nc | nc |
| stimulated IL-13 | | | | |

*nc: no change, ↑: increase, ↓: decrease

### Lung Inflammatory Cells

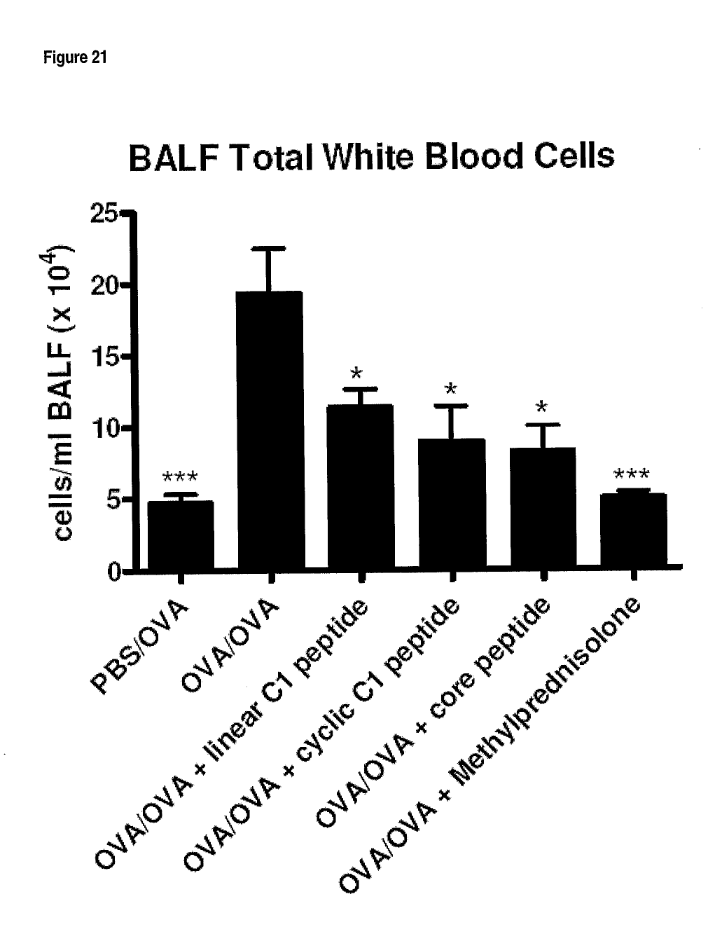

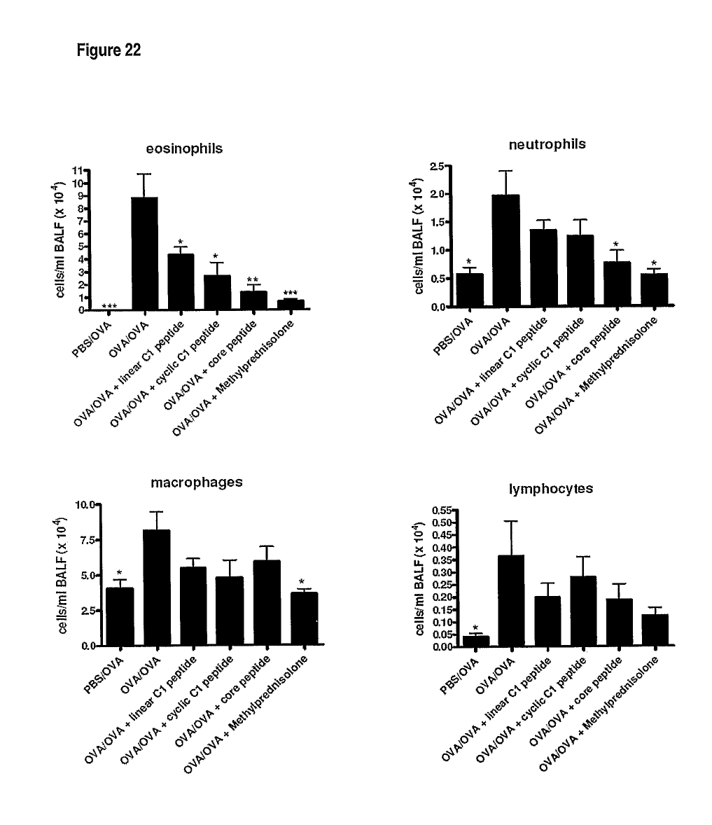

Treatment of allergic mice with either linear C1 peptide, cyclic C1 peptide, core peptide or methylprednisolone significantly decreased total inflammatory cell counts in bronchoalveolar lavage fluid (BALF) (FIG. 21), Upon examination of differential white blood cell counts in BALF it is apparent that the decrease in the total number of cells observed following treatment with linear C1 peptide and cyclic C1 peptide can be attributed to a decrease in eosinophils. Following treatment with core peptide the decrease in the total number of cells observed can be attributed to a reduction in neutrophils and eosinophils. The decrease in the total number of cells observed with methylprednisolone treatment can be attributed to a decrease in neutrophils, eosinophils and macrophages. There is no significant impact of peptide treatment on lymphocyte numbers in the airways (FIG. 22).

### Cytokine Production

Treatment of allergic mice with linear C1 peptide had no significant impact on locally produced (peribronchial lymph node derived) IL-5 or IL-13. Systemic (spleen derived) IL-5 was significantly decreased but no change in systemic IL-13 was observed. Treatment with cyclic C1 peptide led to a significant decrease in IL-5 and IL-13 production both locally and systemically. Treatment with core peptide or methylprednisolone led to a significant decrease in the production of systemic IL-5 and IL-13. Treatment with core peptide also led to a significant decrease in locally produced IL-5 (FIG. 23).

### Airways Hyperreactivity

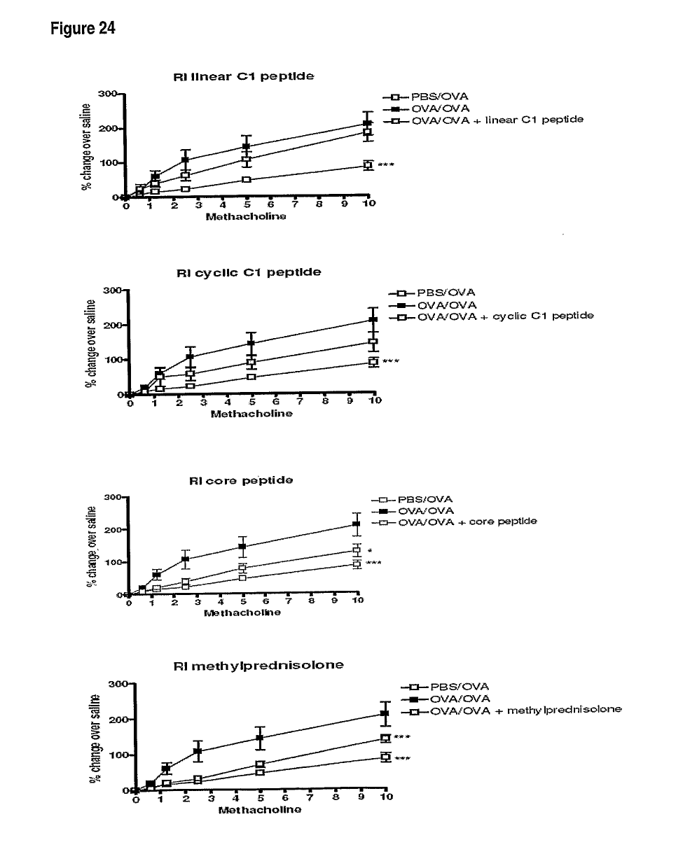

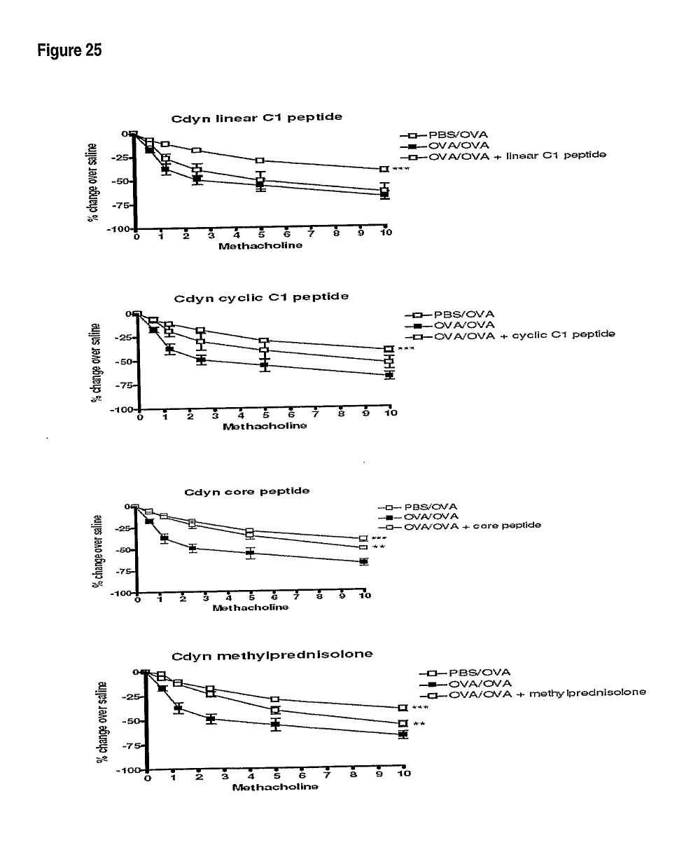

Administration of allergic mice with linear C1 peptide and cyclic C1 peptide had no significant effect on airway resistance (FIG. 24) or dynamic compliance (FIG. 25) in response to methacholine challenge. Mice treated with core peptide and methylprednisolone showed significantly decreased airway resistance and increased dynamic compliance.

### Summary

Treatment of allergic mice with linear C1 peptide, cyclic C1 peptide, core peptide or methylprednisolone resulted in a suppressed immune response as indicated by a decrease in total inflammatory cells in the airways. Treatment with peptides also showed a systemic decrease in the two effector Th2 cytokines (IL-5 and IL-13). Treatment with cyclic C1 peptide also decreased locally produced IL-5 and IL-13. This suppression of the immune response by the peptides was accompanied by changes in airway hyperreactivity. The core peptide and methylprednisolone treatments significantly suppressed the increase in airway hyperreactivity normally observed in this mouse model of allergic airways disease.

The results presented herein indicate that cyclic C1 and similar peptides are effective in ameliorating T-cell based diseases, suppressing immune responses and decreasing inflammation and are thus useful as therapeutics for a range of diseases.

### Example 12

### Treatment of NOD Mice with C1 Peptide

NOD mice were fasted for at least 1.5 h prior to blood glucose level (BGL) measurements.

In the morning, all mice were treated with intraperitoneal (i.p.) injections of 0.1 mg C1 and BGLs were taken. Control mice received 0.2 mL of water. Any mice that displayed a BGL of higher than 15 mM on two consecutive days were deemed diabetic.

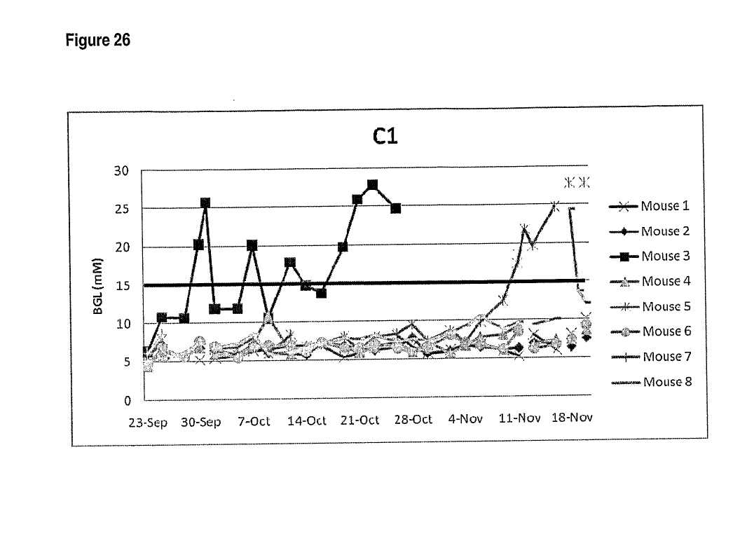

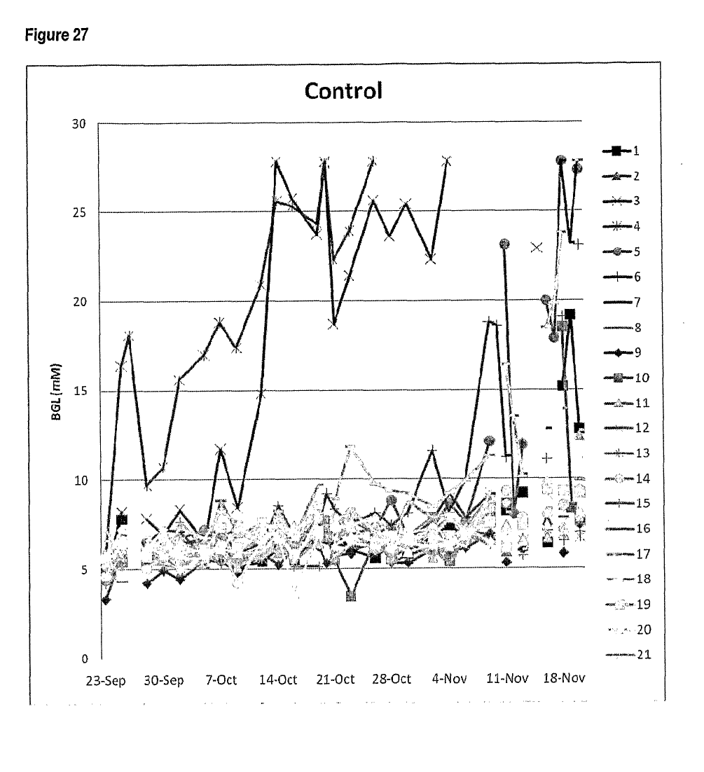

Preliminary results are shown in FIG. 26, where 2/7 C1-treated mice developed diabetes. Mouse 3 developed diabetes within a few days of the commencement of treatment. One other mouse (mouse 5) developed diabetes. In the control group (FIG. 27), 7/21 mice developed diabetes.

In comparing FIGS. 26 and 27 it can be seen that the blood glucose level of mice treated with C1 is more tightly controlled than the control group suggesting a role for cyclic peptides of the invention in control of blood glucose levels.

### Example 13

### Compositions

Molecules and agents of the present invention may be used for the treatment or prevention of various disease states and conditions. Such molecules and agents may be administered alone, although it is more typical that they be administered as a pharmaceutical composition.

In accordance with the best mode of performing the invention provided herein, specific exemplary compositions are outlined below. The following are to be construed as merely illustrative examples of compositions and not as a limitation of the scope of the present invention in any way.

### Example 13(a)

### Composition for Parenteral Administration

A composition for intramuscular injection could be prepared to contain 1 ml sterile buffered water, and 1 mg of a suitable agent or molecule.

Similarly, a composition for intravenous infusion may comprise 250 ml of sterile Ringer's solution, and 5 mg of a suitable agent or molecule.

A composition suitable for administration by injection may also be prepared by mixing 1% by weight of a suitable agent or molecule in 10% by volume propylene glycol and water. The solution is sterilised by filtration.

### Example 13(b)

### Capsule Composition

A composition of a suitable agent or molecule in the form of a capsule may be prepared by filling a standard two-piece hard gelatin capsule with 50 mg of the agent or molecule, in powdered form, 100 mg of lactose, 35 mg of talc and 10 mg of magnesium stearate.

### Example 13(c)

### Eye Drop Composition

A typical composition for delivery as an eye drop is outlined below:

| | | |
|---|---|---|
| Suitable agent or compound | 0.3 | g |
| Methyl Hydroxybenzoate | 0.005 | g |
| Propyl Hydroxybenzoate | 0.06 | g |
| Purified Water about to | 100.00 | ml. |

The methyl and propyl hydroxybenzoates are dissolved in 70 ml purified water at 75° C., and the resulting solution is allowed to cool. The suitable agent or molecule is then added, and the solution sterilised by filtration through a membrane filter (0.22 μm pore size), and aseptically packed into sterile containers.

### Example 13(d)

### Composition for Inhalation Administration

For an aerosol container with a capacity of 20-30 ml: a mixture of 10 mg of a suitable agent or compound with 0.5-0.8% by weight of a lubricating agent, such as polysorbate 85 or oleic acid, is dispersed in a propellant, such as freon, and put into an appropriate aerosol container for either intranasal or oral inhalation administration.

### Example 13(e)

### Ointment Composition

A typical composition for delivery as an ointment includes 1.0 g of a suitable agent or molecule, together with white soft paraffin to 100.0 g, dispersed to produce a smooth, homogeneous product.

### Example 13(f)

### Topical Cream Composition

A typical composition for delivery as a topical cream is outlined below:

| | |
|---|---|
| Suitable agent or molecule | 1.0 g |
| Polawax GP 200 | 25.0 g |
| Lanolin Anhydrous | 3.0 g |
| White Beeswax | 4.5 g |
| Methyl hydroxybenzoate | 0.1 g |
| Deionised & sterilised Water to | 100.0 g |

The polawax, beeswax and lanolin are heated together at 60° C., a solution of methyl hydroxybenzoate is added and homogenisation achieved using high speed stirring. The temperature is then allowed to fall to 50° C. The agent or molecule is then added and dispersed throughout, and the composition is allowed to cool with slow speed stirring.

### REFERENCES

Bender, V., Ali, M., Amon, M., Diefenbach, E. and Manolios, N., J. Biol. Chem. 279, 54002-54007, 2004.
Davis, J. H., Jeffrey, K. R., Bloom, M., Valic, M. I. and Higgs, T. P., Chem. Phys. Lett. 42, 390, 1976.
Gerber, D., Quintana, F. J., Bloch, I., Cohen, I. R. and Shai, Y., FASEB J. 19, 1190-1193, 2005.
Manolios, N., Collier, S., Taylor, J., Pollard, J., Harrison, L. and Bender, V., Nature Med. 3, 84-88, 1997.
Salamon, Z., Brown, M. F. and Tollin, G., Trends Biochem. Sci. 24, 213-219, 1999.
Seelig, J., Biochim. Biophys. Acta 515, 105, 1978.
D. Skropeta, K. A. Jolliffe, P. Turner, Pseudo-prolines as Removable Turn Inducers: Tools for the Cyclisation of Small Peptides. J. Org. Chem., 2004, 69, 8804-8809
Temelkovski, J., S. P. Hogan, et al. (1998). “An improved murine model of asthma: selective airway inflammation, epithelial lesions and increased methacholine responsiveness following chronic exposure to aerosolised allergen.” Thorax 53: 849-856.
Foster, P. S., Y. Ming, et al. (2000). “Dissociation of inflammatory and epithelial responses in a murine model of chronic asthma.” Lab Invest 80: 655-662.
Kelly L. Asquith, Hayley S. Ramshaw, Philip M. Hansbro, Kenneth W. Beagley, Angel F. Lopez and Paul S. Foster J. lmmunol. 2008; 180;1199-1206.

1. A cyclic peptide comprising alternating D- and L-form amino acids, and wherein the peptide possesses immunomodulatory activity.

2. The cyclic peptide of claim 1 wherein the immunomodulatory activity comprises the ability to inhibit T cell activation, proliferation or stimulation.

3. The cyclic peptide of claim 1, comprising
(i) between 6 and 14 amino acids; or
(ii) between 8 and 12 amino acids; or
(iii) 10 amino acids.

4. (canceled)

5. (canceled)

6. The cyclic peptide of claim 1 comprising 10 amino acids in which 5 amino acids are L-form amino acids and 5 amino acids are D-form amino acids.

7. The cyclic peptide of claim 1, having a net positive charge.

8. The cyclic peptide of claim 7 wherein the net positive charge is at least +2.

9. The cyclic peptide of claim 1, comprising two positively charged amino acid residues separated by 1 to 5 hydrophobic residues.

10. The cyclic peptide of claim 9 wherein at least one of the positively charged residues is a lysine residue.

11. The cyclic peptide of claim 1, comprising a lysine residue and a second positively charged residue separated by a spacer of five amino acid residues, wherein the second positively charged residue is an arginine or lysine residue.

12. The cyclic peptide of claim 11 wherein the five amino acid spacer comprises no net charge or a net positive charge.

13. The cyclic peptide of claim 1 comprising a structure as depicted in any one FIGS. 1 to 4, or a cyclic peptide (Cl) of formula 1: 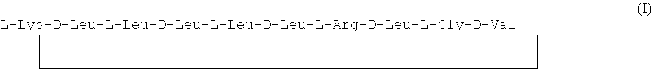
or a cyclic peptide of formula II:

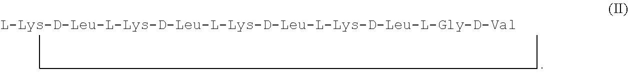

14. (canceled)

15. (canceled)

16. A cyclic peptide selected from the group comprising, L_{D}RL_{D}LL_{D}LL_{D}KV_{D}G, L_{D}R_{D}L_{D}L_{D}L_{D}L_{D}L_{D}K_{D}V_{D}G, LRLLLLLKVG, L_{D}KL_{D}LL_{D}LL_{D}KV_{D}G, LR_{D}LL_{D}LL_{D}LK_{K}VG_{D,} L_{D}LL_{D}RL_{D}LL_{D}KV_{D}G, R_{D}LL_{D}LL_{D}LL_{D}KV_{D}G, L_{D}LL_{D}LL_{D}RL_{D}KV_{D}G, L_{D}RL_{D}KL_{D}LV_{D}LL_{D}G, L_{D}RL_{D}LL_{D}LL_{D}RV_{D}G, L_{D}KL_{D}KL_{D}KL_{D}KV_{D}G, L_{D}RL_{D}LL_{D}KL_{D}KV_{D}G, L_{D}RL_{D}KL_{D}LL_{D}KV_{D}G, L_{D}RL_{D}KL_{D}KL_{D}KV_{D}G, L_{D}KL_{D}KK_{D}KL_{D}KV_{D}G, L_{D}RL_{D}KK_{D}KL_{D}KV_{D}G, L_{D}KL_{D}KK_{D}KK_{D}KV_{D}G, L_{D}RL_{D}KK_{D}KK_{D}KV_{D}G, L_{D}RL_{D}KV_{D}G, L_{D}RL_{D}LL_{D}KV_{D}G, GLRILLLKV, L_{D}SL_{D}RL_{D}LL_{D}LL_{D}KV_{D}G, L_{D}KL_{D}RL_{D}LL_{D}LL_{D}KV_{D}G.

17. A The cyclic peptide of claim 1 or 16 conjugated to one or more chemical moieties.

18. The cyclic peptide of claim 1 or 16 conjugated to one or more chemical moieties wherein conjugation is via linkage at one or more carboxyl, hydroxyl and/or amide groups.

19. The cyclic peptide of claim 1 or 16 conjugated to one or more chemical moieties wherein the chemical moiety is a polypeptide, peptide, lipid, sugar or other chemical compound or a carrier or a therapeutic agent.

20. (canceled)

21. A pharmaceutical composition comprising a peptide of claim 1 or 16.

22. The pharmaceutical composition of claim 21 further comprising one or more pharmaceutically acceptable carriers, adjuvants or diluents.

23. An antibody that selectively binds to a peptide of claim 1 or 16.

24. A method for treating or preventing a disease state in a subject, or for treating or preventing a microbial infection in a subject, said method comprising administering to the subject an effective amount of a peptide of claim 1 or 16.

25. The method of claim 24 wherein the disease
(i) is an auto-immune disease, or
(ii) is neural, endocrinal, skeletal, dermal, gastrointestinal, cardiac, respiratory, vascular, of the immune system or transplantation disease, or
(iii) is a cancer, or
(iv) is, or results from, an infection.

26. (canceled)

27. (canceled)

28. (canceled)

29. The method of any one of claim 25 wherein
(i) the respiratory disease is selected from the group consisting of asthma, chronic eosinophilic pneumonia, COPD (chronic obstructive pulmonary disease), COPD associated with bronchitis, pulmonary emphysema, dyspnea associated or not associated with COPD, COPD characterized by irreversible, progressive airway obstruction, adult respiratory distress syndrome (ARDS), exacerbation of airway hyper-reactivity consequent to drug therapy, airway disease associated with pulmonary hypertension, rhinitis, bronchial oedema, bronchial asthma, pulmonary oedema, anaphylaxis and angioedema; or
(ii) the dermal disease is selected from the group consisting of vitiligo, psoriasis, eczema, Herpes simplex, erythema nodosum, acne vulgaris, erythema, erythema multiforme, neurodermatitis, drug rash, urticaria, contact dermatitis, dermatomycosis and herpes zoster.

30. (canceled)

31. The method of claim 24 wherein said disease state is a disease state in which the inhibition of T-cell activation, proliferation or function is desirable.

32. The method of claim 31 wherein the inhibition of T-cell activation, proliferation or function involves the inhibition of IL-2 production in T-cells.

33. (canceled)

34. Use of a peptide of claim 1 or 16 for the manufacture of a medicament for the treatment or prevention of a disease state in a subject or for the treatment or prevention of a microbial infection in a subject.

35. (canceled)

36. The method of claim 24 wherein said disease state is a disease state in which the control of blood glucose levels is desirable.

37. The method of claim 36 wherein the disease state is diabetes.
